(19) United States
(12) Patent Application Publication
(10) Pub. No.: US 2014/0148443 A1
(43) Pub. Date: May 29, 2014

(54) Novel Imidazolines as dual inhibitors of MDM2 and MDMX

(71) Applicant: Hoffmann-La Roche Inc., Nutley, NJ (US)

(72) Inventors: Xin-Jie Chu, Shanghai (CN); Allen John Lovey, North Caldwell, NJ (US); Binh Thanh Vu, North Caldwell, NJ (US); Chunlin Zhao, Livingston, NJ (US)

(73) Assignee: Hoffmann-La Roche Inc., Nutley, NJ (US)

(21) Appl. No.: 14/084,681

(22) Filed: Nov. 20, 2013

Related U.S. Application Data
(60) Provisional application No. 61/730,638, filed on Nov. 28, 2012.

(51) Int. Cl. C07D 233/22; C07D 401/06; C07D 471/10; C07D 498/08; C07D 405/12; C07D 403/12; C07D 401/14; C07D 403/06; C07D 409/12

(52) U.S. Cl. CPC C07D 233/22; C07D 403/06; C07D 401/06; C07D 471/10; C07D 409/12; C07D 405/12; C07D 403/12; C07D 401/14; C07D 498/08
USPC 514/218; 544/370; 514/254.05; 546/210; 514/326; 544/121; 514/235.8; 546/16; 514/278; 514/254.07; 548/314.7; 514/402; 544/357; 514/252.11; 540/492; 544/105; 514/230.5

(57) ABSTRACT

Disclosed are compounds of Formula I

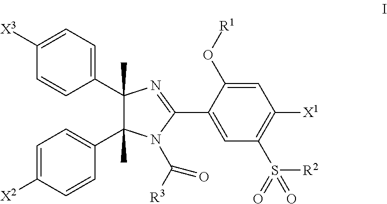

or pharmaceutically acceptable salts thereof, wherein X¹, X², X³, R¹, R² and R³ are as described in this application, and methods of using the compounds in the treatment of cancer.

### PRIORITY TO RELATED APPLICATION

This application claims the benefit of U.S. Provisional Application Ser. No. 61/730,638 filed Nov. 28, 2012.

### FIELD OF THE INVENTION

The present invention relates to imidazoline compounds which act as dual inhibitors of MDM2 and MDMX. These molecules are useful in the amelioration, treatment or control of cancer, especially solid tumors.

### BACKGROUND OF THE INVENTION

Cancer is a disease of uncontrolled cell growth causing local expansion of a tumor and, potentially, distant metastases. One mechanism by which cancer cells grow is by avoidance of apoptosis, or programmed cell death. Alterations in apoptotic pathways have been linked to cancer cells being resistant to standard treatments, e.g., chemotherapeutics or radiation, and to the incidence and progression of cancer. See, e.g., E. Dean et al., “X-linked inhibitor of apoptosis protein as a therapeutic target,” Expert Opin. Ther. Targets (2007) 11(11):1459-1471

p53 is a tumor suppressor protein that plays a central role in protection against development of cancer. It guards cellular integrity and prevents the propagation of permanently damaged clones of cells by the induction of growth arrest or apoptosis. At the molecular level, p53 is a transcription factor that can activate a panel of genes implicated in the regulation of cell cycle and apoptosis. p53 is a potent cell cycle inhibitor which is tightly controlled by its negative regulators: MDM2 and MDMX. MDM2, MDMX and p53 form a feedback control loop. MDM2 and MDMX can bind p53 and inhibit its ability to transactivate p53-regulated genes. MDM2 inhibitors have shown effective p53 activation followed by cell cycle arrest and tumor regression in cancer cells with MDM2 gene amplification. However, their apoptotic activity is marginal in many tumor cell lines expressing normal levels of MDM2 but elevated levels of MDMX. Therefore, antagonizing the binding of both MDM2 and MDMX to p53 is expected to restore p53 apoptotic activity better and across more cancers than inhibiting either regulator alone. See, e.g., B Graves, et al., Activation of the p53 pathway by small-molecule-induced MDM2 and MDMX dimerization, www.pnas.org/cgi/doi/10.1073/pnas.1203789109; and F. Mancini et al., MDM4(MDMX) localizes at the mitochondria and facilitates the p53-mediated intrinsic-apoptotic pathway, The EMBO Journal (2009) 28, 1926-1939

### SUMMARY OF THE INVENTION

One aspect of the present invention is a compound of Formula I

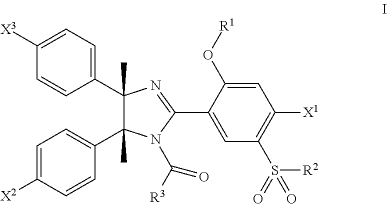

or pharmaceutically acceptable salts thereof, wherein and are as described in this application.

The present invention also relates to pharmaceutical compositions comprising one or more compounds of the invention, or a pharmaceutically acceptable salt thereof, and a pharmaceutically acceptable carrier or excipient.

The present invention further relates to a method of ameliorating, controlling or treating cancer, including specifically solid tumors, for example lung, pancreatic, colon, breast, bone and prostate cancers in a mammal, specifically a human, comprising administering to said mammal a therapeutically effective amount of a compound according to the invention or a pharmaceutically acceptable salt thereof.

### DETAILED DESCRIPTION OF THE INVENTION

### Definitions

Unless otherwise defined, all technical and scientific terms used herein have the same meaning as commonly understood by one of ordinary skill in the art to which this invention belongs.

As used herein, the following terms shall have the following definitions.

“Alkyl” means a monovalent linear or branched saturated hydrocarbon of 1 to 12 carbon atoms. In particular embodiments, alkyl has 1 to 6 carbon atoms, and in more particular embodiments 1 to 4 carbon atoms. As used herein, “lower alkyl” denotes an alkyl group having from 1-6 carbon atoms. Examples of alkyl include methyl, ethyl, propyl, isopropyl, butyl (also known as n-butyl), iso-butyl, sec-butyl, tert-butyl, pentyl, hexyl, and the like. The alkyl group can be optionally enriched in deuterium, e.g., —CD₃, —CD₂CD₃ and the like.

“Aryl” means a monovalent aromatic carbocyclic mono-, bi- or tricyclic ring system comprising 6 to 19 carbon ring atoms. Examples of aryl moieties include, but are not limited to, phenyl, naphthyl (also known as naphthalenyl), tolyl, xylyl, pyridinyl, quinolinyl, pyrimidinyl, imidazolyl, thiazolyl, anthracenyl, tetrazolyl, and fluorenyl.

“Cycloalkyl” means a substituted on unsubstituted stable monovalent saturated monocyclic, bicyclic or tricyclic system which consists of 3 to 10 ring carbon atoms. In particular embodiments, cycloalkyl denotes a monovalent saturated monocyclic hydrocarbon group of 3 to 8 ring carbon atoms. Particular cycloalkyl groups are monocyclic. Examples for monocyclic cycloalkyl are cyclopropyl, cyclobutnyl, cyclopentyl, cyclohexyl or cycloheptyl. Bicyclic means consisting of two saturated carbocycles having one or more carbon atoms in common. Examples for bicyclic cycloalkyl are bicyclo[2.2.1]heptanyl, or bicyclo[2.2.2]octanyl. Tricyclic means consisting of three saturated carbocycles having two or more carbon atoms in common. Examples of tricyclic cycloalkyl include adamantane.

“Halogen” or “Halo” means at atom selected from F, Cl, Br or I. In particular embodiments Halogen means F and Cl.

“Heteroatom” means at atom selected from N, O or S.

“Heteroaryl” means a substituted or unsubstituted aromatic heterocyclic ring system containing up to two rings, at least one ring of which includes 1, 2, or 3 heteroatoms, the remaining ring atoms being carbon. Examples of heteroaryl groups include, but are not limited to, thienyl (also known as thiophenyl), furyl (also known as furanyl), indolyl, pyrrolyl, pyridinyl, pyrazinyl, oxazolyl, isoxazolyl, oxadiazolyl, thiazolyl, quinolinyl, isoquinolinyl, indazolyl, pyrimidinyl, imidazolyl, triazolyl, tetrazolyl, triazinyl, pyrazolyl, benzo[d]isoxazolyl, benzothiazolyl, 2-oxo-2H-chromen-4-yl, benzo[d]isoxazolyl, benzothiophenyl, benzoimidazolyl, naphthyridinyl and cinnolinyl.

In the case of a heteroaryl that is bicyclic it should be understood that one ring may be aryl while the other is heteroaryl and both may be independently substituted or unsubstituted.

“Heterocyclyl,” “heterocycle” or “heterocyclic ring” means a substituted or unsubstituted monovalent saturated or partly unsaturated mono- or bicyclic ring, non-aromatic hydrocarbon system of 3 to 9 ring atoms, comprising 1, 2, or 3 ring heteroatoms selected from N, O and S, the remaining ring atoms being carbon. In particular embodiments, heterocycloalkyl is a monovalent saturated monocyclic ring system of 4 to 7 ring atoms, comprising 1, 2, or 3 ring heteroatoms selected from N, O and S, the remaining ring atoms being carbon. Examples for monocyclic saturated heterocycloalkyl are aziridinyl, oxiranyl, azetidinyl, oxetanyl, pyrrolidinyl, tetrahydrofuranyl, tetrahydro-thiophenyl (also known as tetrahydro-thienyl), pyrazolidinyl, imidazolidinyl, oxazolidinyl, isoxazolidinyl, thiazolidinyl, piperidinyl, tetrahydropyranyl, tetrahydrothiopyranyl, tetrahydrofuranyl, piperazinyl, morpholinyl, thiomorpholinyl, 1,1-dioxo-thiomorpholin-4-yl, azepanyl, diazepanyl, homopiperazinyl, or oxazepanyl. Examples of partly unsaturated heterocycloalkyl are dihydrofuryl, imidazolinyl, dihydro-oxazolyl, dihydro-oxadiazolyl, dihydro-triazolyl, tetrahydro-pyridinyl, tetrahydro-triazinyl or dihydropyranyl.

In the case of a heterocycle that is bicyclic it should be understood that one ring may be heterocycle while the other is cycloalkyl, and either or both may be independently substituted. Examples for bicyclic saturated heterocycloalkyl are 8-aza-bicyclo[3.2.1]octyl, 8-oxa-3-aza-bicyclo[3.2.1]octyl, quinuclidinyl, 9-aza-bicyclo[3.3.1]nonyl, 3-oxa-9-aza-bicyclo[3.3.1]nonyl, or 3-thia-9-aza-bicyclo[3.3.1]nonyl.

“IC₅₀” refers to the concentration of a particular compound required to inhibit 50% of a specific measured activity. IC₅₀ can be measured, inter alia, as is described subsequently in Example 66.

“Oxo” or (“Oxy”) means ═O.

“Pharmaceutically acceptable,” such as pharmaceutically acceptable carrier, excipient, etc., means pharmacologically acceptable and substantially non-toxic to the subject to which the particular compound is administered.

“Pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, trifluoroacetic acid and the like. Sample base-addition salts include those derived from ammonium, potassium, sodium and, quaternary ammonium hydroxides, such as for example, tetramethylammonium hydroxide. Chemical modification of a pharmaceutical compound (i.e. drug) into a salt is a technique well known to pharmaceutical chemists to obtain improved physical and chemical stability, hygroscopicity, flowability and solubility of compounds. See, e.g., Ansel et al., Pharmaceutical Dosage Forms and Drug Delivery Systems (1995) at pgs. 456-457.

“Substituted,” as in substituted alkyl, aryl or heteroaryl means that the substitution (i.e. replacement of one hydrogen atom) can occur at one or more positions and, unless otherwise indicated, that the substituents at each substitution site are independently selected from the specified options. The term “optionally substituted” refers to the fact that one or more hydrogen atoms of a chemical group (with one or more hydrogen atoms) can be, but does not necessarily have to be, substituted with another substituent. The term “spirally substituted” when referring to two, means that the rings have one atom in common. An example of such substitution is piperidine spirally substituted with peperidinyl which is diaza-spiro[5.5]undecane.

The definitions described herein apply irrespective of whether the terms in question appear alone or in combination. It is contemplated that the definitions described herein can be appended to form chemically-relevant combinations, such as e.g. “heterocycloalkylaryl”, “haloalkylheteroaryl”, “arylalkylheterocycloalkyl”, or “alkoxyalkyl”. The last member of the combination is the radical which is binding to the rest of the molecule. The other members of the combination are attached to the binding radical in reversed order in respect of the literal sequence, e.g. the combination arylalkylheterocycloalkyl refers to a heterocycloalkyl-radical which is substituted by an alkyl which is substituted by an aryl.

As used in this application, if a formula or group appears to be missing a substituent, that is it appears the valence is not complete, it is presumed the missing substituent is an H.

In the structural formulae presented herein a broken bond (a) denotes that the substituent is below the plane of the paper and a wedged bond (b) denotes that the substituent is above the plane of the paper.

In one embodiment, the present invention relates to compounds of Formula I

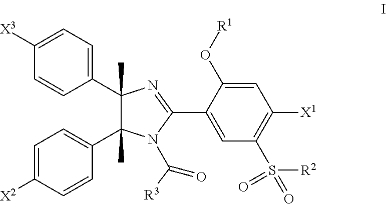

wherein,
X¹, X², X³ are the same or each is independently selected from halogen;
R¹ is lower alkyl that optionally is substituted with halogen;
R² is selected from the group

a) lower alkyl,

b) heterocycle, and

c) NR⁶R⁷;

R₃ is selected from the group:

a) -2-piperazinone that optionally is substituted with lower alkyl that optionally is substituted with OH,

b) a substituted piperazine of the formula II

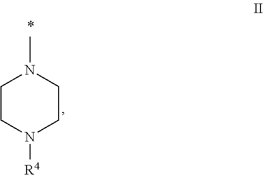

c) an optionally substituted piperidine of formula III

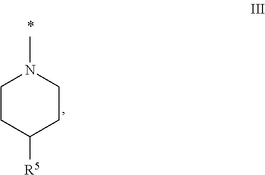

d) an optionally substituted pyrrolidynyl of formula IV

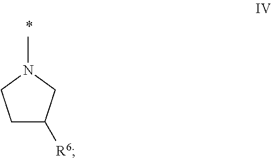

and

e) an optionally substituted piperidine of formula V

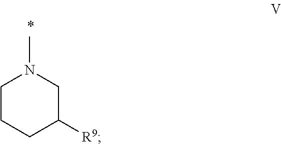

R⁴ is selected from the group:

a) H,

b) lower alkyl that optionally is substituted with
1) OR⁸,
2) C(O)NR⁶R⁷,
3) SO₂R⁸,
4) C(O)-heterocyclyl,
5) C(O)OR⁶,
6) NHSO₂R⁸,
7) NHC(O)R⁸, and
8) CF₃,

c) SO₂R⁸,

d) heterocyclyl substituted with oxo, and

e) C(O)R⁸;

R⁵ is selected from the group

a) H

b) lower alkyl that optionally is substituted with C(O)NR⁶R⁷, heterocyclyl

and SO₂R⁸,

c) NR⁶R⁷,

e) NHC(O)NHR⁸,

f) NHC(O)-heterocyclyl,

g) NHSO₂R⁸

h) OR⁸,

i) heterocyclyl that optionally is substituted with oxo, and

j) piperidynyl which is spirally attached to the piperidine of formula III and which optionally is substituted with lower alkyl-C(O)NR⁶R⁷ or lower alkyl-SO₂R⁸;

R⁶ and R⁷ are the same or each is independently selected from

a) H,

b) lower alkyl that optionally is substituted with
1) O-lower alkyl,
2) N(CH₃)₂, and
3) heterocyclyl,

c) C(O)-lower alkyl; and

d) NR⁶R⁷—NR⁶R⁷;

R⁸ is selected from the group

a) H,

b) lower alkyl that optionally is substituted with OH and heteroaryl,

c) cycloalkyl, and

d) heterocyclyl that optionally is substituted with oxo; and

R⁹ is selected from H and OH;
or a pharmaceutically acceptable salt thereof.

One embodiment of the invention relates to compounds of Formula I wherein X¹, X² and X³ are chloro, or a pharmaceutically acceptable salt of said compound.

Another embodiment of the invention relates to compounds of Formula I wherein R¹ is ethyl or isopropyl, or a pharmaceutically acceptable salt of said compound.

Another embodiment of the invention relates to compounds of Formula I wherein R² is lower alkyl, or a pharmaceutically acceptable salt of said compound. In a particular embodiment R² is selected from methyl and ethyl.

Another embodiment of the invention relates to compounds of Formula I wherein R² is heterocycle, or a pharmaceutically acceptable salt of said compound. In a particular embodiment, R² is selected from pyrrolidine, morpholine and 8-oxa-3-aza-bicyclo[3.2.1]octane, more particularly pyrrolidine.

Another embodiment of the invention relates to compounds of Formula I wherein R² is NR⁶R⁷, and R⁶ and R⁷ are as defined above, or a pharmaceutically acceptable salt of said compound. In a particular embodiment, R² is selected from NH₂ and tert-butylamine

Another embodiment of the invention relates to compounds of Formula I wherein R³ is a piperazine of formula II and R⁴ is a defined above, or a pharmaceutically acceptable salt of said compound. In a particular embodiment R⁴ is lower alkyl that is substituted with C(O)-heterocycle and the heterocycle is selected from 4-morpholinyl, 1-pyrrolidinyl and 1-piperidinyl.

In another embodiment R³ is a piperazine of formula II and R⁴ is lower alkyl that is substituted with C(O)NR⁶R⁷, or a pharmaceutically acceptable salt of said compound. In a specific embodiment NR⁶R⁷ is selected from NH₂ and N(CH₃)₂.

In another embodiment R³ is a piperazine of formula II and R⁴ is lower alkyl that is substituted with SO₂R⁸. In a particular embodiment R⁸ is lower alkyl, more particularly CH₃ and CH₂CH₃.

Another embodiment of the invention relates to compounds of Formula I wherein R³ is a piperazine of formula II and R⁴ is SO₂R⁸, or a pharmaceutically acceptable salt of said compound. In a particular embodiment R⁸ is lower alkyl, specifically methyl.

Another embodiment of the invention relates to compounds of Formula I wherein R³ is a piperazine of formula II and R⁴ is heterocycle that optionally is substituted with oxo, or a pharmaceutically acceptable salt of said compound. In a particular embodiment, R⁴ is 3-tetrahydro-thiophenyl-1,1-dioxide.

Another embodiment of the invention relates to compounds of Formula I wherein R³ is a piperazine of formula II and R⁴ is C(O)R⁸, or a pharmaceutically acceptable salt of said compound. In a particular embodiment R⁸ is lower alkyl substituted by heteroaryl.

Another embodiment of the invention relates to compounds of Formula I wherein R³ is a piperidine of formula III and R⁵ is as defined above, or a pharmaceutically acceptable salt of said compound. In a particular embodiment R⁵ is lower alkyl that optionally is substituted with a group selected from C(O)NR⁶R⁷, SO₂R⁸ and heterocyclyl. In a specific embodiment, R⁶ and R⁷ are both H. In another specific embodiment R⁸ is lower alkyl. In another specific embodiment the heterocyclyl is selected from morpholinyl, pyrrolidinyl and peperidynyl.

Another embodiment of the invention relates to compounds of Formula I wherein R³ is a piperidine of formula III and R⁵ is NR⁶R⁷, or a pharmaceutically acceptable salt of said compound. In a particular embodiment R⁵ is NH₂.

Another embodiment of the invention relates to compounds of Formula I wherein R³ is a piperidine of formula III and R⁵ is NHC(O)NHR⁸, or a pharmaceutically acceptable salt of said compound. In a particular embodiment R⁸ is lower alkyl, more particularly R⁸ is selected from ethyl and propyl.

Another embodiment of the invention relates to compounds of Formula I wherein R³ is a piperidine of formula III and R⁵ is NH(C)O-heterocycle, or a pharmaceutically acceptable salt of said compound. In a particular embodiment the heterocycle is pyrrolidinyl.

Another embodiment of the invention relates to compounds of Formula I wherein R³ is a piperidine of formula III and R⁵ is NHSO₂R⁸, or a pharmaceutically acceptable salt of said compound. In a particular embodiment R⁸ is lower alkyl, more particularly methyl.

Another embodiment of the invention relates to compounds of Formula I wherein R³ is a piperidine of formula III and R⁵ is OR⁸, or a pharmaceutically acceptable salt of said compound. In a particular embodiment R⁸ is H.

Another embodiment of the invention relates to compounds of Formula I wherein R³ is a piperidine of formula III and R⁵ is heterocycle that optionally is substituted with oxo, or a pharmaceutically acceptable salt of said compound. In a particular embodiment R⁵ is 5-oxo-[1,4]diazepam-1-yl.

Another embodiment of the invention relates to compounds of Formula I wherein R³ is a piperidine of formula III and R⁵ is piperidynyl that is spirally attached to the piperidine of formula III and which optionally is substituted with —CH₂C(O)NH₂ or —(CH₂)₂SO₂CH₂CH₃, or a pharmaceutically acceptable salt of said compound.

Another embodiment of the invention relates to compounds of Formula I wherein R³ is a pyrrolidynyl of formula IV and R⁶ is as defined above, or a pharmaceutically acceptable salt of said compound. In a particular embodiment R⁶ is —N(lower alkyl)₂-N(lower alkyl)₂.

Another embodiment of the invention relates to compounds of Formula I wherein R³ is an optionally substituted piperidine of formula V and R⁹ is OH, or a pharmaceutically acceptable salt of said compound.

Another embodiment of the invention relates to compounds of Formula I wherein X¹, X² and X³ are chloro, R¹ is ethyl or isopropyl, R² is lower alkyl or NR⁶R⁷, and R³ is an optionally substituted piperazine of Formula II, or a pharmaceutically acceptable salt of said compound. In an embodiment R⁴ is lower alkyl that optionally is substituted as defined above. In another embodiment, R⁴ is SO₂R⁸.

Another embodiment of the invention relates to compounds of Formula I wherein X¹, X² and X³ are chloro, R¹ is ethyl or isopropyl, R² is lower alkyl or NR⁶R⁷, and R³ is an optionally substituted piperidine of Formula III, or a pharmaceutically acceptable salt of said compound. In an embodiment, R⁵ is lower alkyl that optionally is substituted with C(O)NR⁶R⁷ or SO₂R⁸. In another embodiment, R⁵ is NHSO₂R⁸.

Another embodiment of the invention relates to compounds of Formula I wherein X¹, X² and X³ are chloro, R¹ is ethyl or isopropyl, R² is lower alkyl or NR⁶R⁷, and R³ is an optionally substituted pyrrolidynyl of Formula IV, or a pharmaceutically acceptable salt of said compound. In an embodiment R⁶ is N(lower alkyl)₂-N(lower alkyl)₂.

Another embodiment of the invention relates to compounds of Formula I wherein X¹, X² and X³ are chloro, R¹ is ethyl or isopropyl, R² is lower alkyl or NR⁶R⁷, and R³ an optionally substituted piperidine of Formula V, or a pharmaceutically acceptable salt of said compound. In an embodiment R⁹ is OH.

Another embodiment of the invention relates to compounds of Formula I wherein X¹, X² and X³ are chloro, R¹ is isopropyl, R² is methyl or NH-tert butyl, and R³ is an optionally substituted piperazine of Formula II, or a pharmaceutically acceptable salt of said compound. In an embodiment R⁴ is lower alkyl that optionally is substituted with SO₂R⁸ and R⁸ is methyl.

Another embodiment of the invention relates to compounds of Formula I wherein X¹, X² and X³ are chloro, R¹ is isopropyl, R² is methyl or pyrrolidine, and R³ is an optionally substituted piperidine of Formula III, and R⁵ is methyl that is substituted with C(O)NH₂, or a pharmaceutically acceptable salt of said compound.

Compounds according to the invention wherein R³ is an optionally substituted piperazine of Formula II include:
4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperazin-2-one (Example 2);
[(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-[4-((S)-2,3-dihydroxy-propyl)-piperazin-1-yl]-methanone (Example 3);
4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-1-((S)-3,4-dihydroxy-butyl)-piperazin-2-one (Example 4);
2-{4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperazin-1-yl}-acetamide (Example 6);
[(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-(4-methanesulfonyl-piperazin-1-yl)-methanone (Example 7);
[(4S,5R)-2-(4-Chloro-5-ethanesulfonyl-2-ethoxy-phenyl)-4,5-bis-(4-chloro-phenyl)-4,5-dimethyl-4,5-dihydro-imidazol-1-yl]-[4-(3-methanesulfonyl-propyl)-piperazin-1-yl]-methanone (Example 12);
[(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-[4-(3-ethanesulfonyl-propyl)-piperazin-1-yl]-methanone (Example 14);
2-{4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperazin-1-yl}-N,N-bis-(2-methoxy-ethyl)-acetamide (Example 15);
2-{4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperazin-1-yl}-N,N-bis-(2-ethoxy-ethyl)-acetamide (Example 16);
2-{4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperazin-1-yl}-1-morpholin-4-yl-ethanone (Example 17);
[(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-[4-(2-methanesulfonyl-ethyl)-piperazin-1-yl]-methanone (Example 18);
[(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-[4-(1,1-dioxo-tetrahydro-thiophen-3-yl)-piperazin-1-yl]-methanone (Example 20);
2-{4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperazin-1-yl}-N,N-bis-(2-isopropoxy-ethyl)-acetamide (Example 23);
[(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-[4-(2-ethanesulfonyl-ethyl)-piperazin-1-yl]-methanone (Example 26);
3-{4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperazin-1-yl}-propionamide (Example 27);
2-{4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperazin-1-yl}-N-methyl-acetamide (Example 29);
{4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperazin-1-yl}-acetic acid, hydrochloride (Example 35);
[(4S,5R)-2-(4-Chloro-2-isopropoxy-5-methanesulfonyl-phenyl)-4,5-bis-(4-chloro-phenyl)-4,5-dimethyl-4,5-dihydro-imidazol-1-yl]-[4-(3-methanesulfonyl-propyl)-piperazin-1-yl]-methanone (Example 40);
[(4S,5R)-2-(4-Chloro-2-cyclopropylmethoxy-5-methanesulfonyl-phenyl)-4,5-bis-(4-chloro-phenyl)-4,5-dimethyl-4,5-dihydro-imidazol-1-yl]-[4-(3-methanesulfonyl-propyl)-piperazin-1-yl]-methanone (Example 42);
[(4S,5R)-2-[4-Chloro-2-(2-fluoro-ethoxy)-5-methanesulfonyl-phenyl]-4,5-bis-(4-chloro-phenyl)-4,5-dimethyl-4,5-dihydro-imidazol-1-yl]-[4-(3-methanesulfonyl-propyl)-piperazin-1-yl]-methanone (Example 43);
[(4S,5R)-2-(2-sec-Butoxy-4-chloro-5-methanesulfonyl-phenyl)-4,5-bis-(4-chloro-phenyl)-4,5-dimethyl-4,5-dihydro-imidazol-1-yl]-[4-(3-methanesulfonyl-propyl)-piperazin-1-yl]-methanone (Example 44);
5-{(4S,5R)-4,5-Bis-(4-chloro-phenyl)-1-[4-(3,4-dihydroxy-butyl)-piperazine-1-carbonyl]-4,5-dimethyl-4,5-dihydro-1H-imidazol-2-yl}-N-tert-butyl-2-chloro-4-ethoxy-benzenesulfonamide (Example 45);
5-((4S,5R)-4,5-Bis-(4-chloro-phenyl)-1-{4-[2-(2-hydroxy-ethoxy)-ethyl]-piperazine-1-carbonyl}-4,5-dimethyl-4,5-dihydro-1H-imidazol-2-yl)-N-tert-butyl-2-chloro-4-ethoxy-benzenesulfonamide (Example 46);
5-((4S,5R)-4,5-Bis-(4-chloro-phenyl)-4,5-dimethyl-1-{4-[2-(4-methyl-piperazin-1-yl)-2-oxo-ethyl]-piperazine-1-carbonyl}-4,5-dihydro-1H-imidazol-2-yl)-N-tert-butyl-2-chloro-4-ethoxy-benzenesulfonamide (Example 47);
2-{4-[(4S,5R)-2-(5-tert-Butylsulfamoyl-4-chloro-2-ethoxy-phenyl)-4,5-bis-(4-chloro-phenyl)-4,5-dimethyl-4,5-dihydro-imidazole-1-carbonyl]-piperazin-1-yl}-N-(tetrahydro-furan-2-ylmethyl)-acetamide (Example 48);
2-{4-[(4S,5R)-2-(5-tert-Butylsulfamoyl-4-chloro-2-ethoxy-phenyl)-4,5-bis-(4-chloro-phenyl)-4,5-dimethyl-4,5-dihydro-imidazole-1-carbonyl]-piperazin-1-yl}-N-(2-methoxy-ethyl)-acetamide (Example 49);
3-{4-[(4S,5R)-2-(5-tert-Butylsulfamoyl-4-chloro-2-ethoxy-phenyl)-4,5-bis-(4-chloro-phenyl)-4,5-dimethyl-4,5-dihydro-imidazole-1-carbonyl]-piperazin-1-yl}-propionamide (Example 50);
5-{(4S,5R)-4,5-Bis-(4-chloro-phenyl)-1-[4-(1,1-dioxo-tetrahydro-thiophen-3-yl)-piperazine-1-carbonyl]-4,5-dimethyl-4,5-dihydro-1H-imidazol-2-yl}-N-tert-butyl-2-chloro-4-ethoxy-benzenesulfonamide (Example 51);
5-{(4S,5R)-4,5-Bis-(4-chloro-phenyl)-4,5-dimethyl-1-[4-(2-tetrazol-1-yl-acetyl)-piperazine-1-carbonyl]-4,5-dihydro-1H-imidazol-2-yl}-N-tert-butyl-2-chloro-4-ethoxy-benzenesulfonamide (Example 52);
{(4S,5R)-4,5-Bis-(4-chloro-phenyl)-2-[4-chloro-2-propoxy-5-(pyrrolidine-1-sulfonyl)-phenyl]-4,5-dimethyl-4,5-dihydro-imidazol-1-yl}-[4-(3-methanesulfonyl-propyl)-piperazin-1-yl]-methanone (Example 55);
[(4S,5R)-2-[4-Chloro-2-ethoxy-5-(morpholine-4-sulfonyl)-phenyl]-4,5-bis-(4-chloro-phenyl)-4,5-dimethyl-4,5-dihydro-imidazol-1-yl]-[4-(3-methanesulfonyl-propyl)-piperazin-1-yl]-methanone (Example 57);
[(4S,5R)-2-[4-Chloro-2-isopropoxy-5-(pyrrolidine-1-sulfonyl)-phenyl]-4,5-bis-(4-chloro-phenyl)-4,5-dimethyl-4,5-dihydro-imidazol-1-yl]-[4-(3-methanesulfonyl-propyl)-piperazin-1-yl]-methanone (Example 58);
[(4S,5R)-2-{4-Chloro-2-isopropoxy-5-[(1R,5S)-(8-oxa-3-aza-bicyclo[3.2.1]oct-3-yl)sulfonyl]-phenyl}-4,5-bis-(4-chloro-phenyl)-4,5-dimethyl-4,5-dihydro-imidazol-1-yl]-[4-(3-methanesulfonyl-propyl)-piperazin-1-yl]-methanone (Example 62);
5-{(4S,5R)-4,5-Bis-(4-chloro-phenyl)-1-[4-(3-methanesulfonyl-propyl)-piperazine-1-carbonyl]-4,5-dimethyl-4,5-dihydro-1H-imidazol-2-yl}-N-tert-butyl-2-chloro-4-isopropoxy-benzenesulfonamide (Example 63); and
-{(4S,5R)-4,5-Bis-(4-chloro-phenyl)-1-[4-(3-methanesulfonyl-propyl)-piperazine-1-carbonyl]-4,5-dimethyl-4,5-dihydro-1H-imidazol-2-yl}-2-chloro-4-isopropoxy-benzenesulfonamide (Example 65);
   or a pharmaceutically acceptable salt of any of the foregoing compounds.

Compounds according to the invention wherein R³ is an optionally substituted piperidine of Formula III include:
2-{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-acetamide (Example 5);
(4-Amino-piperidin-1-yl)-[(4S,5R)-2-(4-chloro-2-ethoxy-5-methanesulfonyl-phenyl)-4,5-bis-(4-chloro-phenyl)-4,5-dimethyl-4,5-dihydro-imidazol-1-yl]-methanone (Example 8);
[(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-(4-methanesulfonylmethyl-piperidin-1-yl)-methanone (Example 9);
1-{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-3-ethyl-urea (Example 10);
1-{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-3-isopropyl-urea (Example 11);
[(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-[4-(2-methanesulfonyl-ethyl)-piperidin-1-yl]-methanone (Example 13);
[(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-(3,9-diaza-spiro[5.5]undec-3-yl)-methanone (Example 19);
2-{9-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-3,9-diaza-spiro[5.5]undec-3-yl}-acetamide (Example 21);
[(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-[4-(3-methanesulfonyl-propyl)-piperidin-1-yl]-methanone (Example 22);
2-{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-N,N-bis-(2-methoxy-ethyl)-acetamide (Example 24);
2-{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-N,N-bis-(2-ethoxy-ethyl)-acetamide (Example 25);
[(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-[9-(2-ethanesulfonyl-ethyl)-3,9-diaza-spiro[5.5]undec-3-yl]-methanone (Example 28);
Pyrrolidine-1-carboxylic acid {1-[(4S,5R)-2-(4-chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-amide (Example 30);
1-{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-3-cyclopentyl-urea (Example 31);
{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-urea (Example 32);
N-{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-methanesulfonamide (Example 33);
1-{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-3-(1,1-dioxo-tetrahydro-1λ6-thiophen-3-yl)-urea (Example 34);
N-{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-acetamide (Example 36);
[(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-(4-hydroxy-piperidin-1-yl)-methanone (Example 38);
2-{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-acetamide (Example 39);
2-{1-[(4S,5R)-2-(4-Chloro-2-isopropoxy-5-methanesulfonyl-phenyl)-4,5-bis-(4-chloro-phenyl)-4,5-dimethyl-4,5-dihydro-imidazole-1-carbonyl]-piperidin-4-yl}-acetamide (Example 41);
5-{(4S,5R)-4,5-Bis-(4-chloro-phenyl)-4,5-dimethyl-1-[4-(5-oxo-[1,4]diazepan-1-yl)-piperidine-1-carbonyl]-4,5-dihydro-1H-imidazol-2-yl}-N-tert-butyl-2-chloro-4-ethoxy-benzenesulfonamide (Example 53);
2-{1-[(4S,5R)-2-[4-Chloro-2-ethoxy-5-(pyrrolidine-1-sulfonyl)-phenyl]-4,5-bis-(4-chloro-phenyl)-4,5-dimethyl-4,5-dihydro-imidazole-1-carbonyl]-piperidin-4-yl}-acetamide (Example 56);
2-{1-[(4S,5R)-2-[4-Chloro-2-isopropoxy-5-(pyrrolidine-1-sulfonyl)-phenyl]-4,5-bis-(4-chloro-phenyl)-4,5-dimethyl-4,5-dihydro-imidazole-1-carbonyl]-piperidin-4-yl}-acetamide (Example 59);
2-{1-[(4S,5R)-2-(5-tert-Butylsulfamoyl-4-chloro-2-isopropoxy-phenyl)-4,5-bis-(4-chloro-phenyl)-4,5-dimethyl-4,5-dihydro-imidazole-1-carbonyl]-piperidin-4-yl}-acetamide (Example 60);
2-{1-[(4S,5R)-2-{4-Chloro-2-isopropoxy-5-[(1R,5S)-(8-oxa-3-aza-bicyclo[3.2.1]oct-3-yl)sulfonyl]-phenyl}-4,5-bis-(4-chloro-phenyl)-4,5-dimethyl-4,5-dihydro-imidazole-1-carbonyl]-piperidin-4-yl}-acetamide (Example 61); and
2-{1-[(4S,5R)-2-(4-Chloro-2-isopropoxy-5-sulfamoyl-phenyl)-4,5-bis-(4-chloro-phenyl)-4,5-dimethyl-4,5-dihydro-imidazole-1-carbonyl]-piperidin-4-yl}-acetamide (Example 64);
   or a pharmaceutically acceptable salt of any of the foregoing compounds.

Compounds according to the invention wherein R³ is an optionally substituted pyrrolidynyl of Formula IV include:
[(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-{3-[(3-dimethylamino-propyl)-methyl-amino]-pyrrolidin-1-yl}-methanone (Example 37); or a pharmaceutically acceptable salt thereof.

Compounds according to the invention wherein R³ is an optionally substituted piperidine of Formula V include:
5-[(4S,5R)-4,5-Bis-(4-chloro-phenyl)-1-(3-hydroxy-piperidine-1-carbonyl)-4,5-dimethyl-4,5-dihydro-1H-imidazol-2-yl]-N-tert-butyl-2-chloro-4-ethoxy-benzenesulfonamide (Example 54);
   or a pharmaceutically acceptable salt thereof.

Another embodiment of the invention relates to a compound selected from:
[(4S,5R)-2-(4-Chloro-2-isopropoxy-5-methanesulfonyl-phenyl)-4,5-bis-(4-chloro-phenyl)-4,5-dimethyl-4,5-dihydro-imidazol-1-yl]-[4-(3-methanesulfonyl-propyl)-piperazin-1-yl]-methanone (Example 40);
2-{1-[(4S,5R)-2-(4-Chloro-2-isopropoxy-5-methanesulfonyl-phenyl)-4,5-bis-(4-chloro-phenyl)-4,5-dimethyl-4,5-dihydro-imidazole-1-carbonyl]-piperidin-4-yl}-acetamide (Example 41);
2-{1-[(4S,5R)-2-[4-Chloro-2-isopropoxy-5-(pyrrolidine-1-sulfonyl)-phenyl]-4,5-bis-(4-chloro-phenyl)-4,5-dimethyl-4,5-dihydro-imidazole-1-carbonyl]-piperidin-4-yl}-acetamide (Example 59); and
5-{(4S,5R)-4,5-Bis-(4-chloro-phenyl)-1-[4-(3-methanesulfonyl-propyl)-piperazine-1-carbonyl]-4,5-dimethyl-4,5-dihydro-1H-imidazol-2-yl}-N-tert-butyl-2-chloro-4-isopropoxy-benzenesulfonamide (Example 63);
   or a pharmaceutically acceptable salt of any of the foregoing compounds.

The compounds of Formula I as well as their salts have at least one asymmetric carbon atom and therefore may be present as mixtures of different stereoisomers. The various isomers can be isolated by known separation methods, e.g., chromatography.

Compounds disclosed herein and covered by Formula I above may exhibit tautomerism or structural isomerism. It is intended that the invention encompasses any tautomeric or structural isomeric form of these compounds, or mixtures of such forms, and is not limited to any one tautomeric or structural isomeric form depicted in the formulas above.

### Dosages

The compounds of the present invention are thus useful in the amelioration, control or treatment of cell proliferative disorders such as, in particular, oncological disorders. These compounds and formulations containing said compounds are anticipated to be useful in the treatment or control of blood cancers, such as, for example, acute myeloid leukemia, or solid tumors, such as, for example, breast, colon, lung and prostate tumors.

A “therapeutically effective amount” or “effective amount” of a compound in accordance with this invention means an amount of compound that is effective to prevent, alleviate or ameliorate symptoms of disease or prolong the survival of the subject being treated. Determination of a therapeutically effective amount is within the skill in the art.

The therapeutically effective amount or dosage of a compound according to this invention can vary within wide limits and may be determined in a manner known in the art. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 Kg, a daily dosage of about 10 mg to about 10,000 mg, preferably from about 200 mg to about 1,000 mg, should be appropriate, although the upper limit may be exceeded when indicated. The daily dosage can be administered as a single dose or in divided doses, or for parenteral administration, it may be given as one or more bolus injections or as a continuous infusion.

Pharmaceutical preparations useful in the practice of the invention, i.e., comprising the compounds of the invention can be administered internally, such as orally (e.g. in the form of tablets, coated tablets, dragées, hard and soft gelatin capsules, solutions, emulsions or suspensions), nasally (e.g. in the form of nasal sprays) or rectally (e.g. in the form of suppositories). However, the administration can also be effected parentally, such as intramuscularly or intravenously (e.g. in the form of injection solutions). Moreover, administration can be effected topically (e.g. in the form of ointments, creams or oils).

### Compositions/Formulations

In an alternative embodiment, the present invention includes pharmaceutical compositions comprising at least one compound of Formula I, or a pharmaceutically acceptable salt thereof, and a pharmaceutically acceptable excipient and/or carrier.

These pharmaceutical compositions can be suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, as well as the particular mode of administration. The amount of active ingredient which can be combined with a carrier material to produce a single dosage form will generally be that amount of a Formula I compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

The compounds of Formula I and their pharmaceutically acceptable salts and esters can be processed with pharmaceutically inert, inorganic or organic adjuvants for the production of tablets, coated tablets, dragées and hard gelatin capsules. Lactose, polyvinylpyrrolidone, hydroxypropylmethylcellulose, hydroxypropylcellulose, microcrystalline cellulose, corn starch or derivatives thereof, talc, stearic acid or its salts etc. can be used, for example, as such adjuvants for tablets, dragées and hard gelatin capsules.

Suitable adjuvants for soft gelatin capsules, are, for example, vegetable oils, waxes, fats, semi-solid substances and liquid polyols, etc. Suitable adjuvants for the production of solutions and syrups are, for example, H₂O, polyols, saccharose, invert sugar, glucose, etc. Suitable adjuvants for injection solutions are, for example, H₂O, alcohols, polyols, glycerol, vegetable oils, etc. Suitable adjuvants for suppositories are, for example, natural or hardened oils, waxes, fats, semi-solid or liquid polyols, etc. Suitable adjuvants for topical preparations are glycerides, semi-synthetic and synthetic glycerides, hydrogenated oils, liquid waxes, liquid paraffins, liquid fatty alcohols, sterols, polyethylene glycols and cellulose derivatives.

Moreover, the pharmaceutical preparations can contain preservatives, solubilizers, viscosity-increasing substances, stabilizers, wetting agents, emulsifiers, sweeteners, colorants, flavors, salts for varying the osmotic pressure, buffers, masking agents or antioxidants. They can also contain other therapeutic substances.

The compounds in the present invention (compounds of general Formula I) can be prepared using the general reaction scheme set out in the schemes below.

### Synthesis

The compounds of the present invention can be prepared according to schemes 1-3. Briefly, the process involves the formation of imidazoline 5 either by coupling of a tetrasubstituted 1,2-diamine 2 with an acid chloride 3 to form a monoamide derivative 4 followed by cyclodehydration (scheme 1) with phosphorus oxychloride or by reaction of a tetrasubstituted 1,2-diamine 2 with an aromatic ester 6 in the presence of trimethylaluminum (procedure described by Moormann, A. E. et al J. Med. Chem. 1990, 33, 614-626, scheme 2).

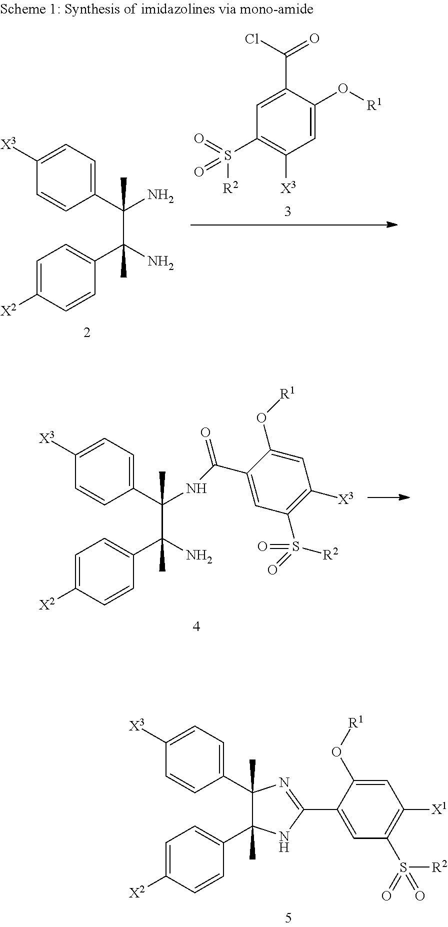

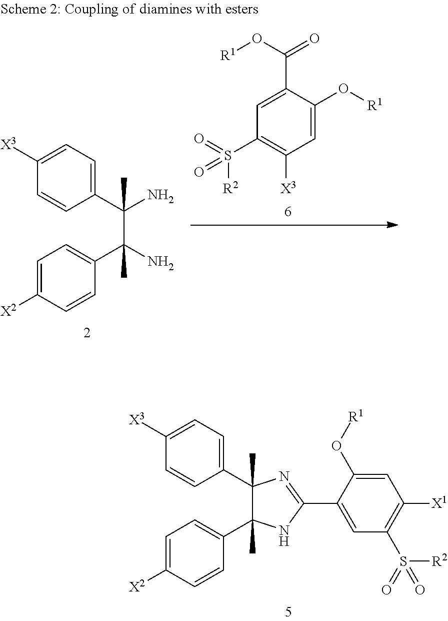

Treatment of the imidazoline 5 with phosgene in the presence of a base such as triethylamine or diisopropylethylamine gives the racemic carbamoyl chloride 8 (scheme 3). Coupling of the racemic carbamoyl chloride 7 with appropriate R amine groups provides the compounds of the formula I as racemic mixtures. Many R amine groups are commercially available. If it is desired, R amine groups can be prepared using synthetic methods known in the art.

If it is desired to prepare the optically active compounds of formula I, the enantiomers of the racemic carbamoyl chloride 7 can be separated using chiral chromatography. Coupling of the desired enantiomer 7A with appropriate R₃ amine groups provides the optically active compounds of the formula I. Also the optically active compounds of formula I can be obtained by chiral separation of the racemic mixtures of 1.

The absolute stereochemistry of the preferred enantiomer of 1 is determined based on the crystal structure of its complex with the human MDM2 (Vassilev et al. Science, 2004, 303, 844-848.

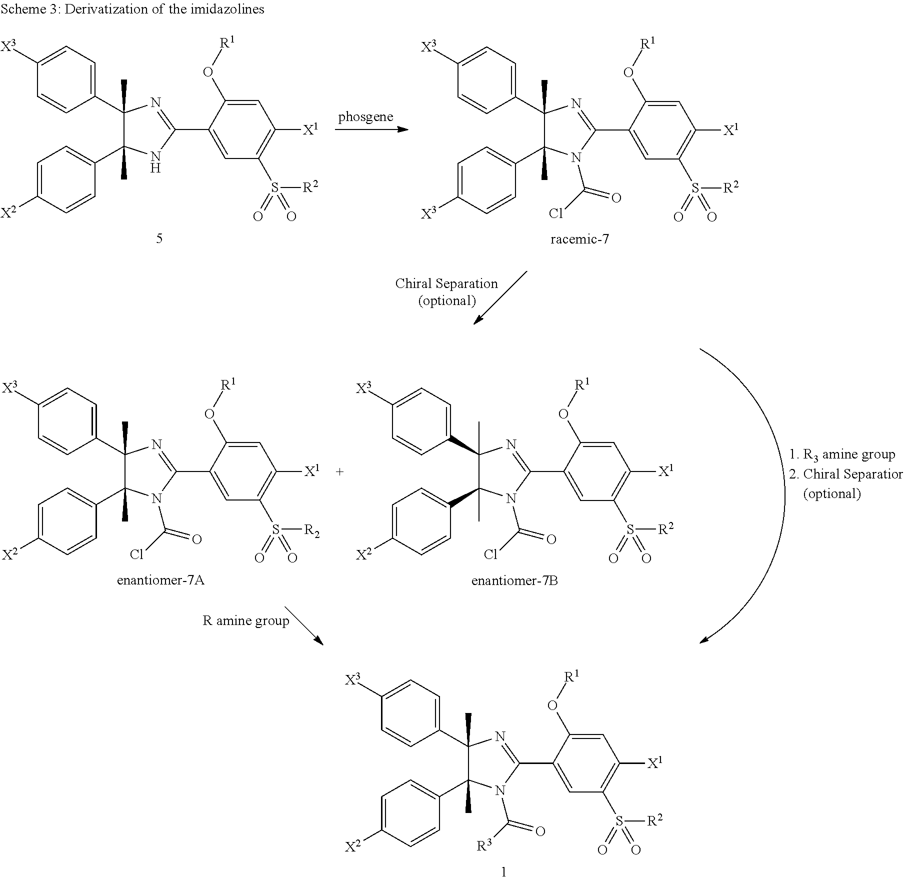

Methods to perform the above described reactions and processes would be apparent to those of ordinary skill in the art based on the present disclosure, or can be deduced in analogy from the examples. Starting materials are commercially available or can be made by methods analogous to those described in the Examples below.

### Crystal Forms

When the compounds of the invention are solids, it is understood by those skilled in the art that these compounds, and their salts, may exist in different crystal or polymorphic forms, all of which are intended to be within the scope of the present invention and specified formulas.

### EXAMPLES

The compounds of the present invention may be synthesized according to known techniques. The following examples and references are provided to aid the understanding of the present invention. The examples are not intended, however, to limit the invention, the true scope of which is set forth in the appended claims. The names of the reactants and final products in the examples were generated using AutoNom 2000 Add-in v4.0 SP2 (function in ISIS Draw, Elsevier/MDL), or AutoNom 2000 TT v4.01.305 (Elsevier/MDL), or functions available in ChemDraw Pro Control 11.0.2 (CambridgeSoft Corp.), or Struct=Name feature of electronic notebooks.

### Example 1

### (4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl

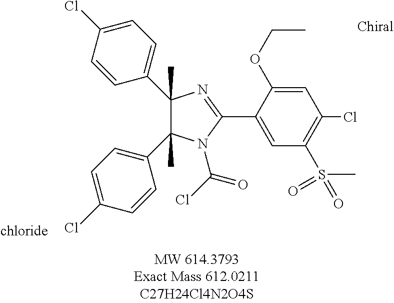 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)Cl]

To neat chlorosulfonic acid (55 mL) was added to 4-chlorosalicylic acid (57.95 mmol) at 0° C. The mixture was stirred at room temperature for 1 h then heated to 50-60° C. for 2 h. After the starting material had disappeared (as confirmed by thin layer chromatography, aliquot of reaction mixture quenched with water/ethyl acetate, developed in 1:1 ethyl acetate:acetonitrile), the reaction mixture was let cooled down to room temperature before pouring into ice dropwise with vigorous stirring. The product was extracted with ethyl ether, and the organic layer was washed with water (2×), brine, dried over magnesium sulfate, filtered and concentrated to give crude 4-chloro-5-chlorosulfonyl-2-hydroxy-benzoic acid (12.4 g). The product was stored in freezer and used without further purification.

To a solution of sodium bicarbonate (5.0 g, 60 mmol) and sodium sulfite (2.4 g, 19 mmol) in water (25 mL) at 70-75° C. was treated with 4-chloro-5-chlorosulfonyl-2-ethoxy-benzoic acid (6.2 g, 21 mmol) in portions according to the procedure described by Imamura, S. et al. (Bioorg. Med. Chem. 2005, 13, 397-416). After 1 h at 75° C., chloroacetic acid (3.0 g, 32 mmol) was carefully added in three portions followed by a solution of aqueous sodium hydroxide (1.28 g, 32 mmol/4 mL water) and stirred at 100° C. for 16 h. This was cooled and poured into 2N hydrochloric acid (100 mL) and extracted with ethyl acetate (2×) The organic extracts were washed with brine, dried over magnesium sulfate, filtered, and concentrated at room temperature to a low volume. This was triturated with ether and filtered to give 4-chloro-2-ethoxy-5-methanesulfonyl-benzoic acid as a colorless solid (5.2 g, 89%).

To a solution of 4-chloro-2-ethoxy-5-methanesulfonyl-benzoic acid (400 mg, 1.44 mmol) in tetrahydrofuran cooled to 0° C. were added 2 drops of dimethylformamide then oxalyl chloride (190 uL, 2.16 mmol). The reaction mixture was stirred at room temperature for 4 h then stored in freezer overnight before being concentrated to give crude 4-chloro-2-ethoxy-5-methanesulfonyl-benzoyl chloride as light yellow oil. It was used without further purification.

To a solution of meso-2,3-bis-(4-chlorophenyl)-2,3-butanediamine (396 mg, 1.27 mmol, prepared as described in Ding et al. U.S. Pat. No. 7,851,626) and diisopropylethylamine (0.6 mL, 3.35 mmol) in tetrahydrofuran (20 mL) cooled to 0° C. was added dropwise a solution of 4-chloro-2-ethoxy-5-methanesulfonyl-benzoyl chloride (400 mg, 1.34 mmol) in tetrahydrofuran. After being stirred at −5° C. for 30 min, the reaction mixture was diluted with iced water and extracted with ethyl acetate (2×50 mL). The combined organic extracts were washed with water, dried over magnesium sulfate, filtered and concentrated. The residue was purified by flash column chromatography (silica gel, eluting with a gradient of 10-30% ethyl acetate in methylene chloride) to give N-[(1S,2R)-2-amino-1,2-bis-(4-chloro-phenyl)-1-methyl-propyl]-4-chloro-2-ethoxy-5-methanesulfonyl-benzamide (415 mg).

To a solution of N-[(1S,2R)-2-amino-1,2-bis-(4-chloro-phenyl)-1-methyl-propyl]-4-chloro-2-ethoxy-5-methanesulfonyl-benzamide (410 mg, 0.72 mmol) in toluene (10 mL) was added phosphorus oxychloride (0.2 mL, 2.16 mmol). The mixture was heated at reflux overnight. Upon cooling to room temperature, the reaction mixture was poured into iced water and basified with saturated solution of sodium bicarbonate and 2N sodium hydroxide. The product was extracted with ethyl acetate (2×30 mL). The combined organic extracts were washed with water, dried over magnesium sulfate, filtered and concentrated. The residue was purified by flash column chromatography (silica gel, eluting with a gradient of 3-8% methanol in methylene chloride) to give (4S,5R)-2-(4-chloro-2-ethoxy-5-methanesulfonyl-phenyl)-4,5-bis-(4-chloro-phenyl)-4,5-dimethyl-4,5-dihydro-imidazole (300 mg).

To a solution of (4S,5R)-2-(4-chloro-2-ethoxy-5-methanesulfonyl-phenyl)-4,5-bis-(4-chloro-phenyl)-4,5-dimethyl-4,5-dihydro-imidazole (300 mg, 0.545 mmol) in methylene chloride (10 mL) cooled to 0° C. were added triethylamine (100 uL, 1.36 mmol) and phosgene (600 uL, 1.10 mmol, 1.9M solution in toluene). The reaction mixture was stirred at 0° C. for 1 h and at room temperature for 30 min before being poured into iced water. The product was extracted with methylene chloride (2×). The combined organic extracts were washed with water, dried over magnesium sulfate, filtered and concentrated. The residue was purified by flash column chromatography (silica gel, eluting with a gradient of 10-30% ethyl acetate in methylene chloride) to give the racemic mixture of (4S,5R)-2-(4-chloro-2-ethoxy-5-methanesulfonyl-phenyl)-4,5-bis-(4-chloro-phenyl)-4,5-dimethyl-4,5-dihydro-imidazole-1-carbonyl chloride as white foam (260 mg). The enantiomers were then separated by supercritical fluid chromatography (Berger Instrument Multi-Gram II, Kromasil OD 3×25 cm, eluting with 15% of acetonitrile in carbon dioxide at 70 mL/min) to give the desired enantiomer (post peak).

### Example 2

### 4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperazin-2-one

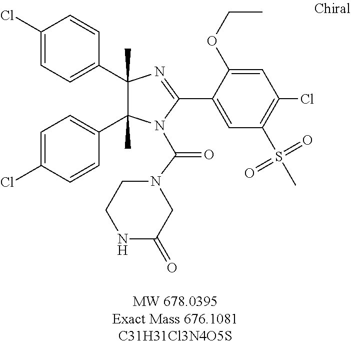 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCNC(=O)C1]

To the suspension of 2-oxo-piperazine (20 mg, 0.2 mmol) in methylene chloride (3 mL) cooled to 0° C. were added triethylamine (45 uL, 0.32 mmol) and a solution of rac-(4S,5R)-2-(4-chloro-2-ethoxy-5-methanesulfonyl-phenyl)-4,5-bis-(4-chloro-phenyl)-4,5-dimethyl-4,5-dihydro-imidazole-1-carbonyl chloride (40 mg, 0.065 mmol, example 1). The mixture was allowed to react for 30 min and then water was added. The product was extracted with methylene chloride (2×20 mL). The organic extracts were dried over anhydrous magnesium sulfate, filtered and concentrated under reduced pressure. Purification by flash column chromatography on silica gel eluting with a gradient of 2-8% methanol in methylene chloride provided 22 mg of rac-4-[(4S,5R)-2-(4-chloro-2-ethoxy-5-methanesulfonyl-phenyl)-4,5-bis-(4-chloro-phenyl)-4,5-dimethyl-4,5-dihydro-imidazole-1-carbonyl]-piperazin-2-one as white solid.

The enantiomers were then separated by supercritical fluid chromatography (Berger Instrument Multi-Gram II, Whelk-O1, eluting with 45% of methanol in carbon dioxide) to give the desired enantiomer (pre peak). HR-MS (ES, m/z) calculated for C₃₁H₃₂Cl₃N₄O₅S [(M+H)⁺] 677.1154, observed 677.1153.

### Example 3

### [(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-[4-((S)-2,3-dihydroxy-propyl)-piperazin-1-yl]-methanone

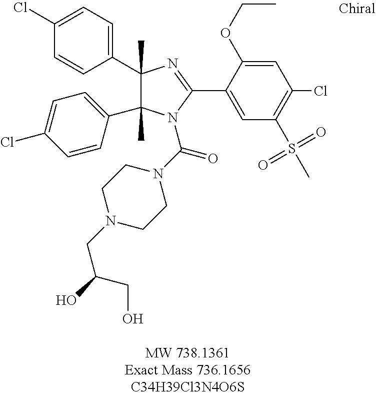 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCN(C[C@H](O)CO)CC1]

In a manner analogous to the method described in Example 2, rac-(4S,5R)-2-(4-chloro-2-ethoxy-5-methanesulfonyl-phenyl)-4,5-bis-(4-chloro-phenyl)-4,5-dimethyl-4,5-dihydro-imidazole-1-carbonyl chloride was reacted with 1-((S)-2,2-dimethyl-[1,3]dioxolan-4-ylmethyl)-piperazine to give rac-[(4S,5R)-2-(4-chloro-2-ethoxy-5-methanesulfonyl-phenyl)-4,5-bis-(4-chloro-phenyl)-4,5-dimethyl-4,5-dihydro-imidazol-1-yl]-[4-((S)-2,3-dihydroxy-propyl)-piperazin-1-yl]-methanone after deprotection of the bis-acetonide intermediate with 2N hydrochloric acid. The enantiomers were then separated by supercritical fluid chromatography (Berger Instrument Multi-Gram II, Whelk-O1, eluting with 40% of methanol in carbon dioxide) to give the desired enantiomer (pre peak). HR-MS (ES, m/z) calculated for C₃₄H₄₀Cl₃N₄O₆S [(M+H)⁺] 737.1729, observed 737.1726.

### Example 4

### 4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-1-((S)-3,4-dihydroxy-butyl)-piperazin-2-one

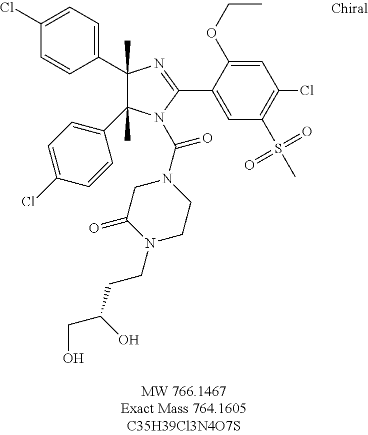 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCN(CC[C@H](O)CO)C(=O)C1]

In a manner analogous to the method described in Example 2, rac-(4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with 1-[2-((S)-2,2-Dimethyl-[1,3]dioxolan-4-yl)-ethyl]-piperazin-2-one to give rac-4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-1-((S)-3,4-dihydroxy-butyl)-piperazin-2-one after deprotection of the bis-acetonide intermediate with 2N hydrochloric acid. The enantiomers were then separated by supercritical fluid chromatography (Berger Instrument Multi-Gram II, Whelk-O1, eluting with 45% of methanol in carbon dioxide) to give the desired enantiomer (pre peak). HR-MS (ES, m/z) calculated for C₃₅H₄₀Cl₃N₄O₇S [(M+H)⁺] 765.1678, observed 765.1683.

### Example 5

### 2-{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-acetamide

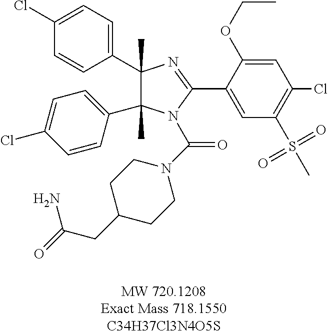 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCC(CC(N)=O)CC1]

In a manner analogous to the method described in Example 2, rac-(4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with piperidin-4-yl-acetamide to give the tittle compound. HR-MS (ES, m/z) calculated for C₃₄H₃₈Cl₃N₄O₅S [(M+H)⁺] 719.1623, observed 719.1625.

### Example 6

### 2-{4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperazin-1-yl}-acetamide

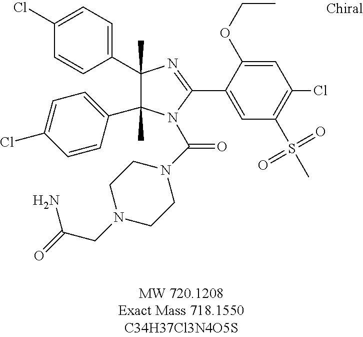 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCN(CC(N)=O)CC1]

In a manner analogous to the method described in example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with piperazin-4-yl-acetamide to give the tittle compound. HR-MS (ES, m/z) calculated for C₃₃H₃₇Cl₃N₅O₅S [(M+H)⁺] 720.1576, observed 720.1579.

### Example 7

### [(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-(4-methanesulfonyl-piperazin-1-yl)-methanone

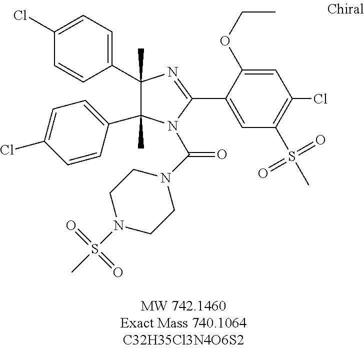 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCN(S(C)(=O)=O)CC1]

In a manner analogous to the method described in example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with 4-methanesulfonyl-piperazine to give the tittle compound. HR-MS (ES, m/z) calculated for C₃₂H₃₆Cl₃N₄O₆S₂ [(M+H)+] 741.1137, observed 741.1132.

### Example 8

### (4-Amino-piperidin-1-yl)-[(4S,5R)-2-(4-chloro-2-ethoxy-5-methanesulfonyl-phenyl)-4,5-bis-(4-chloro-phenyl)-4,5-dimethyl-4,5-dihydro-imidazol-1-yl]-methanone

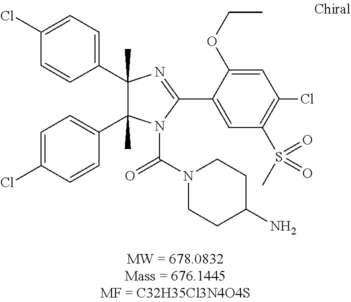 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCC(N)CC1]

In a manner analogous to the method described in Example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with 4-amino-piperidine to give the tittle compound. HR-MS (ES, m/z) calculated for C₃₂H₃₆Cl₃N₄O₄S [(M+H)⁺] 677.1518, observed 677.1517.

### Example 9

### [(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-(4-methanesulfonylmethyl-piperidin-1-yl)-methanone

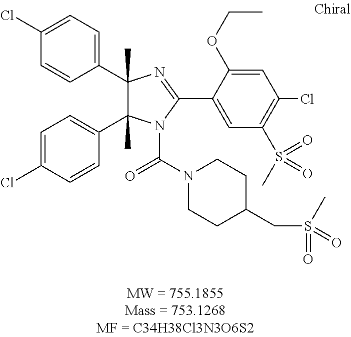 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCC(CS(C)(=O)=O)CC1]

In a manner analogous to the method described in Example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with 4-methanesulfonylmethyl-piperidine to give the tittle compound. HR-MS (ES, m/z) calculated for C₃₄H₃₉Cl₃N₃O₆S₂ [(M+H)⁺] 754.1341, observed 754.1340.

### Example 10

### 1-{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-3-ethyl-urea

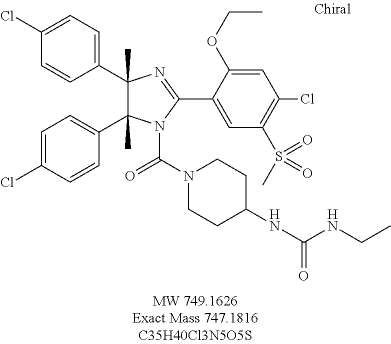 [SMILES: CCNC(=O)NC1CCN(C(=O)N2C(c3cc(S(C)(=O)=O)c(Cl)cc3OCC)=N[C@@](C)(c3ccc(Cl)cc3)[C@@]2(C)c2ccc(Cl)cc2)CC1]

In a manner analogous to the method described in Example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with 1-ethyl-3-piperidin-4-yl-urea to give the tittle compound. HR-MS (ES, m/z) calculated for C₃₅H₄₁Cl₃N₅O₅S [(M+H)⁺] 748.1889, observed 748.1885.

### Example 11

### 1-{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-3-isopropyl-urea

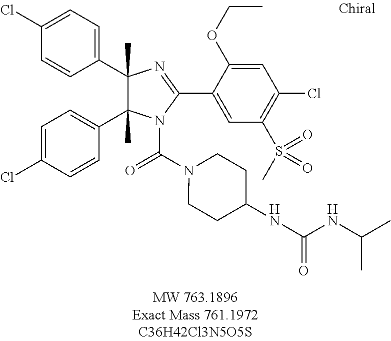 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCC(NC(=O)NC(C)C)CC1]

In a manner analogous to the method described in Example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with 1-isopropyl-3-piperidin-4-yl-urea to give the tittle compound. HR-MS (ES, m/z) calculated for C₃₆H₄₃Cl₃N₅O₅S [(M+H)⁺] 762.2045, observed 762.2046.

### Example 12

### [(4S,5R)-2-(4-Chloro-5-ethanesulfonyl-2-ethoxy-phenyl)-4,5-bis-(4-chloro-phenyl)-4,5-dimethyl-4,5-dihydro-imidazol-1-yl]-[4-(3-methanesulfonyl-propyl)-piperazin-1-yl]-methanone

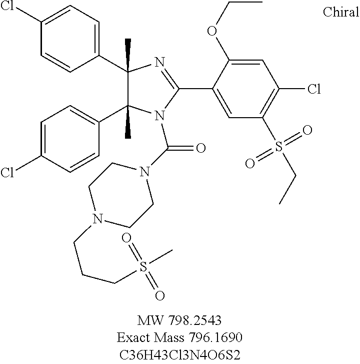 [SMILES: CCOc1cc(Cl)c(S(=O)(=O)CC)cc1C1=N[C@@](C)(c2ccc(Cl)cc2)[C@@](C)(c2ccc(Cl)cc2)N1C(=O)N1CCN(CCCS(C)(=O)=O)CC1]

In a manner analogous to the method described in Example 2, rac-(4S,5R)-2-(4-chloro-5-ethanesulfonyl-2-ethoxy-phenyl)-4,5-bis-(4-chloro-phenyl)-4,5-dimethyl-4,5-dihydro-1H-imidazole-1-carbonyl chloride (prepared in a manner as described for the methylsulfone in example 1) was reacted with 4-(3-methanesulfonyl-propyl)-piperazine (prepared as described in Ding et al. U.S. Pat. No. 7,851,626) to give the tittle compound as a racemic mixture. The enantiomers were then separated by supercritical fluid chromatography (Berger Instrument Multi-Gram II, Whelk-O1, eluting with 45% of methanol in carbon dioxide) to give the desired enantiomer (pre peak). HR-MS (ES, m/z) calculated for C₃₆H₄₄Cl₃N₄O₆S₂ [(M+H)⁺] 797.1763, observed 797.1761.

### Example 13

### [(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-[4-(2-methanesulfonyl-ethyl)-piperidin-1-yl]-methanone

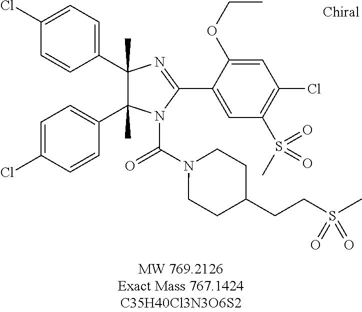 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCC(CCS(C)(=O)=O)CC1]

In a manner analogous to the method described in Example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with 4-(2-methanesulfonyl-ethyl)-piperidine to give the tittle compound. HR-MS (ES, m/z) calculated for C₃₅H₄₁Cl₃N₃O₆S₂ [(M+H)⁺] 768.1497, observed 768.1494.

### Example 14

### [(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-[4-(3-ethanesulfonyl-propyl)-piperazin-1-yl]-methanone

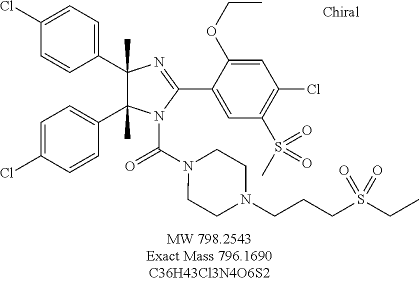 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCN(CCCS(=O)(=O)CC)CC1]

In a manner analogous to the method described in example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with 4-(3-ethanesulfonyl-propyl)-piperazine to give the tittle compound. HR-MS (ES, m/z) calculated for C₃₆H₄₄Cl₃N₄O₆S₂ [(M+H)⁺] 797.1763, observed 797.1762.

### Example 15

### 2-{4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperazin-1-yl}-N,N-bis-(2-methoxy-ethyl)-acetamide

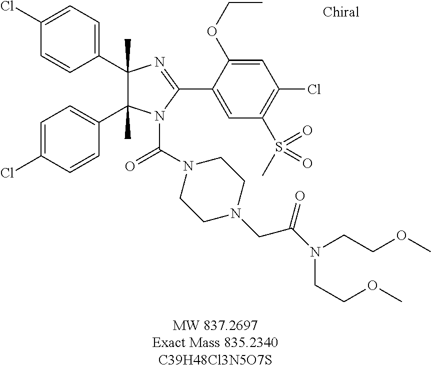 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCN(CC(=O)N(CCOC)CCOC)CC1]

In a manner analogous to the method described in Example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with N,N-bis-(2-methoxy-ethyl)-2-piperazin-1-yl-acetamide to give the tittle compound. HR-MS (ES, m/z) calculated for C₃₉H₄₉Cl₃NO₇S [(M+H)⁺] 836.2413, observed 836.2412.

### Example 16

### 2-{4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperazin-1-yl}-N,N-bis-(2-ethoxy-ethyl)-acetamide

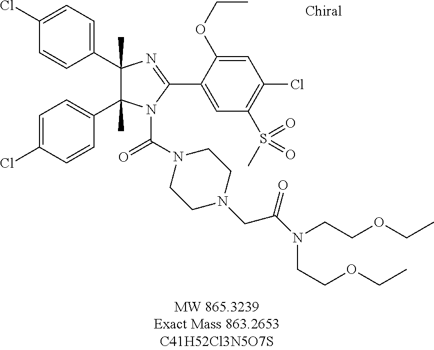 [SMILES: CCOCCN(CCOCC)C(=O)CN1CCN(C(=O)N2C(c3cc(S(C)(=O)=O)c(Cl)cc3OCC)=N[C@@](C)(c3ccc(Cl)cc3)[C@@]2(C)c2ccc(Cl)cc2)CC1]

In a manner analogous to the method described in Example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with N,N-bis-(2-ethoxy-ethyl)-2-piperazin-1-yl-acetamide to give the tittle compound. HR-MS (ES, m/z) calculated for C₄₁H₅₃Cl₃N₅O₇S [(M+H)⁺] 864.2726, observed 864.2728.

### Example 17

### 2-{4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperazin-1-yl}-1-morpholin-4-yl-ethanone

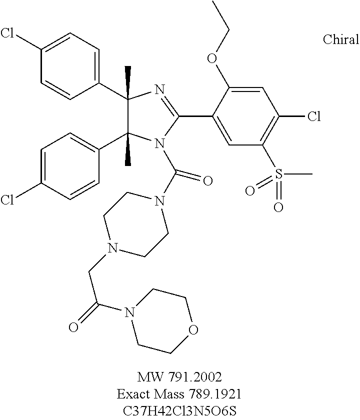 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCN(CC(=O)N2CCOCC2)CC1]

In a manner analogous to the method described in Example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with 1-morpholin-4-yl-2-piperazin-1-yl-ethanone to give the tittle compound. HR-MS (ES, m/z) calculated for C₃₇H₄₃Cl₃N₅O₆S [(M+H)⁺] 790.1994, observed 790.1992.

### Example 18

### [(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-[4-(2-methanesulfonyl-ethyl)-piperazin-1-yl]-methanone

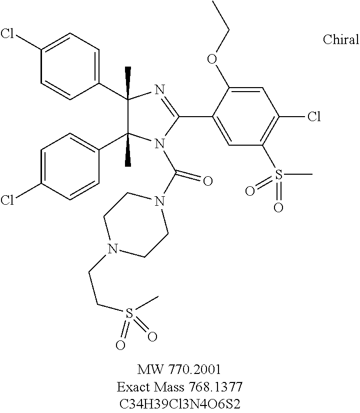 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCN(CCS(C)(=O)=O)CC1]

In a manner analogous to the method described in Example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted 4-(2-methanesulfonyl-ethyl)-piperazine to give the tittle compound. HR-MS (ES, m/z) calculated for C₃₄H₄₀Cl₃N₄O₆S₂ [(M+H)⁺] 769.1450, observed 769.1448.

### Example 19

### [(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-(3,9-diaza-spiro[5.5]undec-3-yl)-methanone

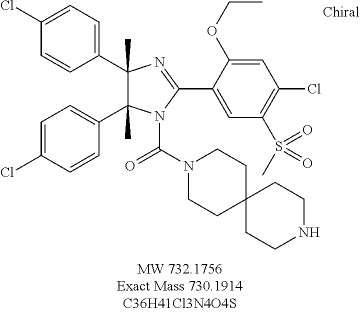 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCC2(CCNCC2)CC1]

In a manner analogous to the method described in example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with phosgene and 3,9-diaza-spiro[5.5]undecane to give the tittle compound. HR-MS (ES, m/z) calculated for C₃₆H₄₂Cl₃N₄O₄S [(M+H)⁺] 731.1987, observed 731.1993.

### Example 20

### [(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-[4-(1,1-dioxo-tetrahydro-thiophen-3-yl)-piperazin-1-yl]-methanone

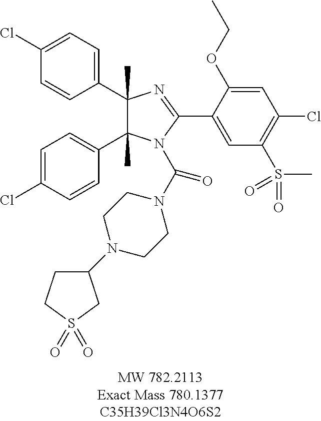 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCN(C2CCS(=O)(=O)C2)CC1]

In a manner analogous to the method described in example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with 4-(1,1-dioxo-tetrahydro-3-thiophenyl)-piperazine to give the tittle compound. HR-MS (ES, m/z) calculated for C₃₅H₄₀Cl₃N₄O₆S₂ [(M+H)⁺] 781.1450, observed 781.1448.

### Example 21

### 2-{9-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-3,9-diaza-spiro[5.5]undec-3-yl}-acetamide

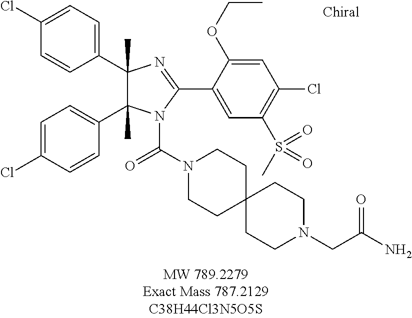 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCC2(CCN(CC(N)=O)CC2)CC1]

In a manner analogous to the method described in Example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with 2-(3,9-diaza-spiro[5.5]undec-3-yl)-acetamide to give the tittle compound. HR-MS (ES, m/z) calculated for C₃₈H₄₅Cl₃N₅O₅S [(M+H)⁺] 788.2202, observed 788.2201.

### Example 22

### [(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-[4-(3-methanesulfonyl-propyl)-piperidin-1-yl]-methanone

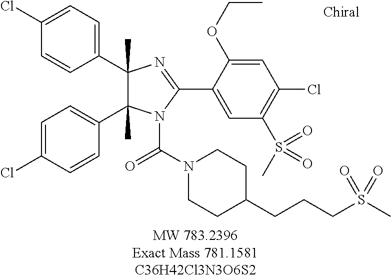 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCC(CCCS(C)(=O)=O)CC1]

In a manner analogous to the method described in Example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with 4-(3-methanesulfonyl-propyl)-piperidine to give the tittle compound. HR-MS (ES, m/z) calculated for C₃₆H₄₃Cl₃N₃O₆S₂ [(M+H)⁺] 782.1654, observed 782.1653.

### Example 23

### 2-{4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperazin-1-yl}-N,N-bis-(2-isopropoxy-ethyl)-acetamide

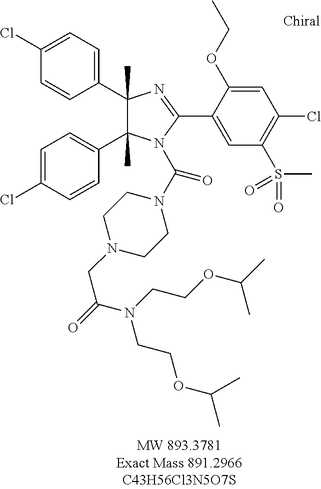 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCN(CC(=O)N(CCOC(C)C)CCOC(C)C)CC1]

In a manner analogous to the method described in Example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with N,N-bis-(2-isopropoxy-ethyl)-2-piperazin-1-yl-acetamide to give the tittle compound. HR-MS (ES, m/z) calculated for C₄₃H₅₇Cl₃N₅O₇S [(M+H)⁺] 892.3039, observed 892.3041.

### Example 24

### 2-{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-N,N-bis-(2-methoxy-ethyl)-acetamide

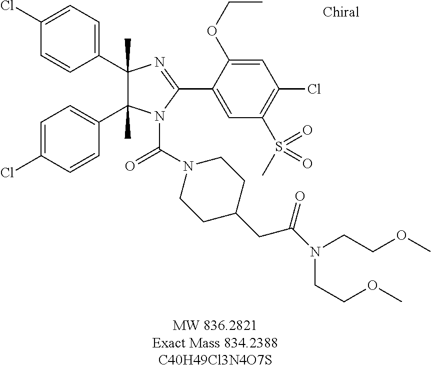 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCC(CC(=O)N(CCOC)CCOC)CC1]

In a manner analogous to the method described in Example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with N,N-bis-(2-methoxy-ethyl)-2-piperidin-4-yl-acetamide to give the tittle compound. HR-MS (ES, m/z) calculated for C₄₀H₅₀Cl₃N₄O₇S [(M+H)⁺] 835.2461, observed 835.2461.

### Example 25

### 2-{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-N,N-bis-(2-ethoxy-ethyl)-acetamide

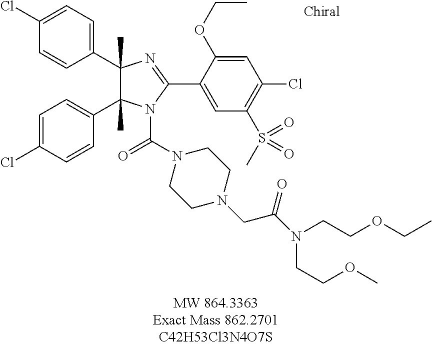 [SMILES: CCOCCN(CCOC)C(=O)CN1CCN(C(=O)N2C(c3cc(S(C)(=O)=O)c(Cl)cc3OCC)=N[C@@](C)(c3ccc(Cl)cc3)[C@@]2(C)c2ccc(Cl)cc2)CC1]

In a manner analogous to the method described in Example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with N,N-bis-(2-ethoxy-ethyl)-2-piperidin-4-yl-acetamide to give the tittle compound. HR-MS (ES, m/z) calculated for C₄₂H₅₄Cl₃N₄O₇S [(M+H)⁺] 863.2774, observed 863.2778.

### Example 26

### [(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-[4-(2-ethanesulfonyl-ethyl)-piperazin-1-yl]-methanone

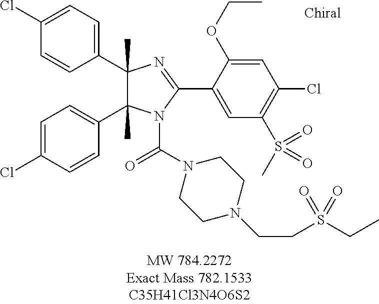 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCN(CCS(=O)(=O)CC)CC1]

In a manner analogous to the method described in Example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with 4-(2-ethanesulfonyl-ethyl)-piperazine to give the tittle compound. HR-MS (ES, m/z) calculated for C₃₅H₄₂Cl₃N₄O₆S₂ [(M+H)⁺] 783.1606, observed 783.1604.

### Example 27

### 3-{4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperazin-1-yl}-propionamide

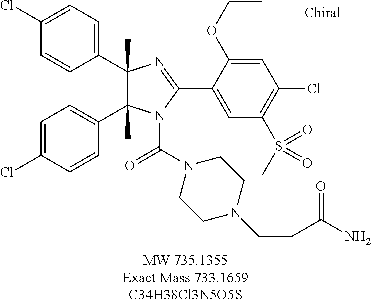 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCN(CCC(N)=O)CC1]

In a manner analogous to the method described in Example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with 3-piperazin-1-yl-propionamide to give the tittle compound. HR-MS (ES, m/z) calculated for C₃₄H₃₉Cl₃N₅O₅S [(M+H)⁺] 734.1732, observed 734.1733.

### Example 28

### [(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-[9-(2-ethanesulfonyl-ethyl)-3,9-diaza-spiro[5.5]undec-3-yl]-methanone

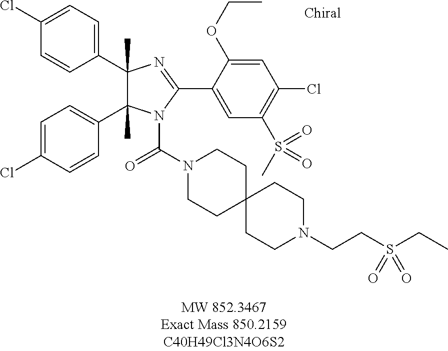 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCC2(CCN(CCS(=O)(=O)CC)CC2)CC1]

In a manner analogous to the method described in Example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with 3-(2-ethanesulfonyl-ethyl)-3,9-diaza-spiro[5.5]undecane to give the tittle compound. HR-MS (ES, m/z) calculated for C₄₀H₅₀Cl₃ N₄O₆S₂ [(M+H)⁺] 851.2232, observed 851.2228.

### Example 29

### 2-{4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperazin-1-yl}-N-methyl-acetamide

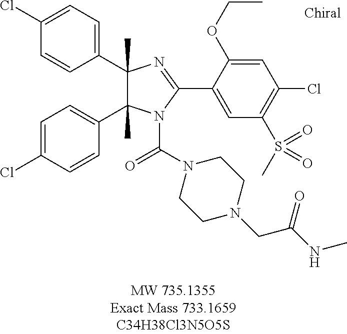 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCN(CC(=O)NC)CC1]

In a manner analogous to the method described in Example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with 4-piperazine-N-methyl-acetamide to give the tittle compound. HR-MS (ES, m/z) calculated for C₃₄H₃₉Cl₃N₅O₅S [(M+H)⁺] 734.1732, observed 734.1732.

### Example 30

### Pyrrolidine-1-carboxylic acid {1-[(4S,5R)-2-(4-chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-amide

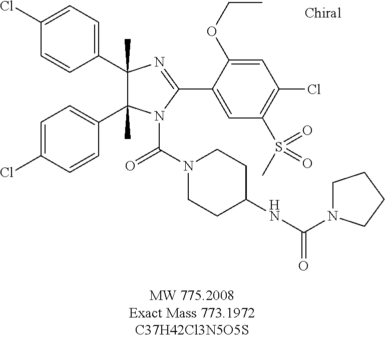 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCC(NC(=O)N2CCCC2)CC1]

In a manner analogous to the method described in Example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with pyrrolidine-1-carboxylic acid piperidin-4-ylamide to give the tittle compound. HR-MS (ES, m/z) calculated for C₃₇H₄₃Cl₃N₅O₅S [(M+H)⁺] 774.2045, observed 774.2049.

### Example 31

### 1-{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-3-cyclopentyl-urea

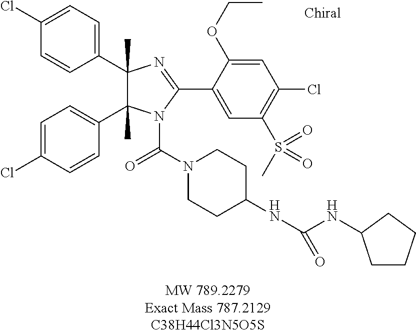 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCC(NC(=O)NC2CCCC2)CC1]

In a manner analogous to the method described in Example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with 1-cyclopentyl-3-piperidin-4-yl-urea to give the tittle compound. HR-MS (ES, m/z) calculated for C₃₈H₄₅Cl₃N₅O₅S [(M+H)⁺] 788.2202, observed 788.2201.

### Example 32

### {1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-urea

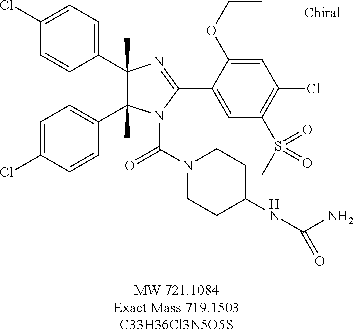 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCC(NC(N)=O)CC1]

In a manner analogous to the method described in example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with piperidin-4-yl-urea to give the tittle compound. HR-MS (ES, m/z) calculated for C₃₃H₃₇Cl₃N₅O₅S [(M+H)⁺] 720.1576, observed 720.1582.

### Example 33

### N-{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-methanesulfonamide

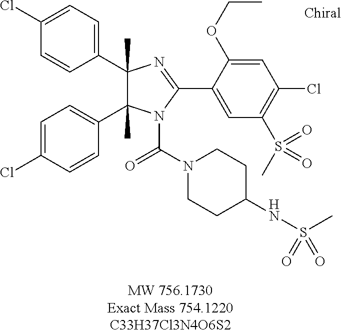 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCC(NS(C)(=O)=O)CC1]

In a manner analogous to the method described in Example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with piperidin-4-yl-methanesulfonamide to give the tittle compound. HR-MS (ES, m/z) calculated for C₃₃H₃₈Cl₃N₄O₆S₂ [(M+H)⁺] 755.1293, observed 755.1295.

### Example 34

### 1-{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-3-(1,1-dioxo-tetrahydro-1λ6-thiophen-3-yl)-urea

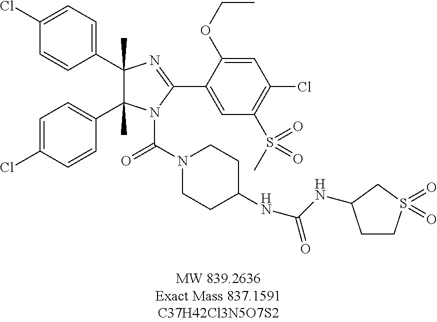 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCC(NC(=O)NC2CCS(=O)(=O)C2)CC1]

In a manner analogous to the method described in example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with 1-(1,1-dioxo-tetrahydro-thiophen-3-yl)-3-piperidin-4-yl-urea to give the tittle compound. HR-MS (ES, m/z) calculated for C₃₇H₄₃Cl₃N₅O₇S₂ [(M+H)⁺] 838.1664, observed 838.1661.

### Example 35

### {4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperazin-1-yl}-acetic acid, hydrochloride

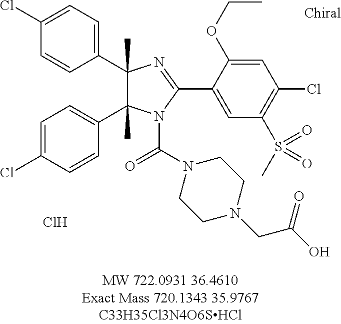 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCN(CC(=O)O)CC1 ; Cl]

In a manner analogous to the method described in Example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with piperazin-1-yl-acetic acid to give the tittle compound. HR-MS (ES, m/z) calculated for C₃₃H₃₆Cl₃N₄O₆S [(M+H)⁺] 721.1416, observed 721.1412.

### Example 36

### N-{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-acetamide

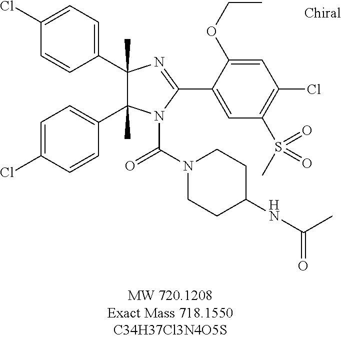 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCC(NC(C)=O)CC1]

In a manner analogous to the method described in Example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with piperidin-4-yl-acetamide to give the tittle compound. HR-MS (ES, m/z) calculated for C₃₄H₃₈Cl₃N₄O₅S [(M+H)⁺] 719.1623, observed 719.1624.

### Example 37

### [(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-{3-[(3-dimethylamino-propyl)-methyl-amino]-pyrrolidin-1-yl}-methanone

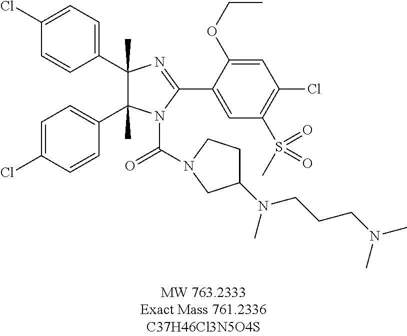 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCC(N(C)CCCN(C)C)C1]

In a manner analogous to the method described in Example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with 3-[(3-dimethylamino-propyl)-methyl-amino]-pyrrolidine to give the tittle compound. HR-MS (ES, m/z) calculated for C₃₇H₄₇Cl₃N₅O₄S [(M+H)⁺] 762.2409, observed 762.2407.

### Example 38

### [(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-(4-hydroxy-piperidin-1-yl)-methanone

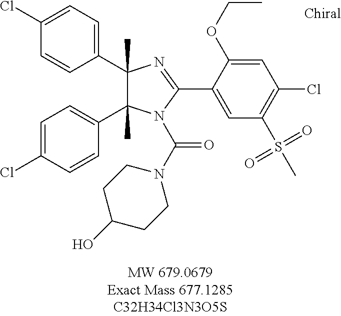 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCC(O)CC1]

In a manner analogous to the method described in example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with 4-hydroxy-piperidine to give the tittle compound. LC-MS (ES, m/z) calculated for C₃₂H₃₅Cl₃N₃O₅S [(M+H)⁺] 678, observed 678.

### Example 39

### 2-{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-acetamide

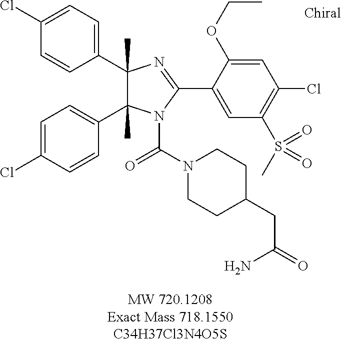 [SMILES: CCOc1cc(Cl)c(S(C)(=O)=O)cc1C1=N[C@@](C)(c2ccc(Cl)cc2)[C@@](C)(c2ccc(Cl)cc2)N1C(=O)N1CCC(CC(N)=O)CC1]

In a manner analogous to the method described in Example 2, (4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with piperidine-4-yl-acetamide to give the tittle compound. HR-MS (ES, m/z) calculated for C₃₄H₃₈Cl₃N₄O₅S [(M+H)⁺] 719.1623, observed 719.1625.

### Example 40

### [(4S,5R)-2-(4-Chloro-2-isopropoxy-5-methanesulfonyl-phenyl)-4,5-bis-(4-chloro-phenyl)-4,5-dimethyl-4,5-dihydro-imidazol-1-yl]-[4-(3-methanesulfonyl-propyl)-piperazin-1-yl]-methanone

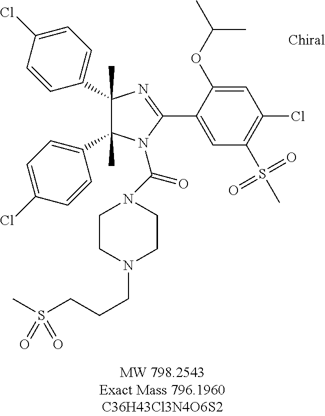 [SMILES: CC(C)Oc1cc(Cl)c(S(C)(=O)=O)cc1C1=N[C@@](C)(c2ccc(Cl)cc2)[C@@](C)(c2ccc(Cl)cc2)N1C(=O)N1CCN(CCCS(C)(=O)=O)CC1]

In a manner analogous to the method described in Example 2, rac-(4S,5R)-2-(4-chloro-2-ethoxy-5-methanesulfonyl-phenyl)-4,5-bis-(4-chloro-phenyl)-4,5-dimethyl-4,5-dihydro-1H-imidazole-1-carbonyl chloride was reacted with 4-(3-methanesulfonyl-propyl)-piperazine hydrochloride (prepared as described in Ding et al. U.S. Pat. No. 7,851,626) to give ((4S,5R)-2-(4-chloro-2-ethoxy-5-(methylsulfonyl)phenyl)-4,5-bis(4-chlorophenyl)-4,5-dimethyl-4,5-dihydro-1H-imidazol-1-yl)(4-(3-(methylsulfonyl)propyl)piperazin-1-yl)methanone. LC-MS (ES, m/z) calculated for C₃₅H₄₁Cl₃N₄O₆S₂ [(M+H)⁺] 783, observed 783.

To solution of ((4S,5R)-2-(4-chloro-2-ethoxy-5-(methylsulfonyl)phenyl)-4,5-bis(4-chlorophenyl)-4,5-dimethyl-4,5-dihydro-1H-imidazol-1-yl)(4-(3-(methylsulfonyl)propyl)-piperazin-1-yl)methanone (520 mg, 0.663 mmol) in methylene chloride (30 mL) cooled to 0° C. was added boron tribromide (2.65 mL, 2.65 mmol, 1M solution in methylene chloride). After 10 min, the ice bath was removed, and the reaction mixture was stirred at room temperature overnight. The reaction mixture was then poured into ice, extract with ethyl acetate (2×). The organic extracts were washed with brine, dried over anhydrous magnesium sulfate, filtered and concentrated. Purification of the crude residue by flash column chromatography (40 g of silica gel, eluting with 50-100% ethyl acetate in hexanes, then with 2-15% methanol in ethyl acetate) provided ((4S,5R)-2-(4-chloro-2-hydroxy-5-(methylsulfonyl)phenyl)-4,5-bis(4-chlorophenyl)-4,5-dimethyl-4,5-dihydro-1H-imidazol-1-yl)(4-(3-(methylsulfonyl)propyl)piperazin-1-yl)methanone as light yellow foam (355 mg, 71% yield). LC-MS (ES, m/z) calculated for C₃₃H₃₈Cl₃N₄O₆S₂ [(M+H)⁺] 755, observed 755.

The mixture of rac-[(4S,5R)-2-(4-chloro-2-hydroxy-5-methanesulfonyl-phenyl)-4,5-bis-(4-chloro-phenyl)-4,5-dimethyl-4,5-dihydro-imidazol-1-yl]-[4-(3-methanesulfonyl-propyl)-piperazin-1-yl]-methanone (250 mg, 0.331 mmol), potassium carbonate (91 mg, 0.662 mmol) and isopropyl iodide (661 uL, 6.61 mmol) in ethanol (50 mL) was heated at 80° C. for 6 h. It was concentrated in vacuo, and the residue was taken in ethyl acetate and washed with water, brine, dried over anhydrous magnesium sulfate, filtered and concentrated in vacuo. Purification of the crude residue by HPLC (C18, eluting with 20-95% acetonitrile-water) gave the title compound as a racemic mixture (166 mg, 63% yield). The enantiomers were then separated by supercritical fluid chromatography (Berger Instrument Multi-Gram II, Kromasil OD 3×25 cm, eluting with 25% of 1:1 acetonitrile:methanol in carbon dioxide) to give the desired enantiomer (post peak). LC-MS (ES, m/z) calculated for C₃₆H₄₄Cl₃N₄O₆S₂ [(M+H)⁺] 797, observed 797.

### Example 41

### 2-{1-[(4S,5R)-2-(4-Chloro-2-isopropoxy-5-methanesulfonyl-phenyl)-4,5-bis-(4-chloro-phenyl)-4,5-dimethyl-4,5-dihydro-imidazole-1-carbonyl]-piperidin-4-yl}-acetamide

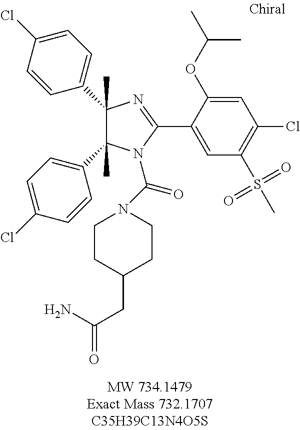 [SMILES: CC(C)Oc1cc(Cl)c(S(C)(=O)=O)cc1C1=N[C@@](C)(c2ccc(Cl)cc2)[C@@](C)(c2ccc(Cl)cc2)N1C(=O)N1CCC(CC(N)=O)CC1]

In a manner analogous to the method described in Example 40, 2-{1-[(4S,5R)-2-(4-chloro-2-isopropoxy-5-methanesulfonyl-phenyl)-4,5-bis-(4-chloro-phenyl)-4,5-dimethyl-4,5-dihydro-imidazole-1-carbonyl]-piperidin-4-yl}-acetamide (Example 5) was converted to the corresponding phenol by reacting with boron tribromide. Alkylation of the phenol with isopropyl iodide gave the title compound as a racemic mixture. The enantiomers were then separated by supercritical fluid chromatography (Berger Instrument Multi-Gram II, Kromasil OD 3×25 cm, eluting with 25% of 1:1 acetonitrile:methanol in carbon dioxide) to give the desired enantiomer (post peak). LC-MS (ES, m/z) calculated for C₃₅H₄₀Cl₃N₄O₅S [(M+H)⁺] 733, observed 733.

### Example 42

### [(4S,5R)-2-(4-Chloro-2-cyclopropylmethoxy-5-methanesulfonyl-phenyl)-4,5-bis-(4-chloro-phenyl)-4,5-dimethyl-4,5-dihydro-imidazol-1-yl]-[4-(3-methanesulfonyl-propyl)-piperazin-1-yl]-methanone

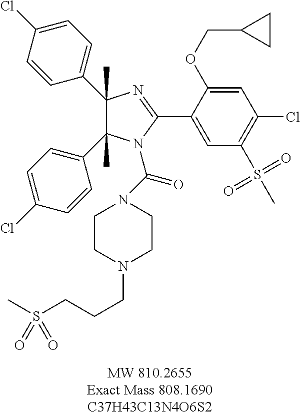 [SMILES: C[C@@]1(c2ccc(Cl)cc2)N=C(c2cc(S(C)(=O)=O)c(Cl)cc2OCC2CC2)N(C(=O)N2CCN(CCCS(C)(=O)=O)CC2)[C@]1(C)c1ccc(Cl)cc1]

In a manner analogous to the method described in Example 40, rac-[(4S,5R)-2-(4-chloro-2-hydroxy-5-methanesulfonyl-phenyl)-4,5-bis-(4-chloro-phenyl)-4,5-dimethyl-4,5-dihydro-imidazol-1-yl]-[4-(3-methanesulfonyl-propyl)-piperazin-1-yl]-methanone was alkylated with (bromomethyl)cyclopropane to give the title compound. LC-MS (ES, m/z) calculated for C₃₇H₄₄Cl₃N₄O₆S₂ [(M+H)⁺] 809, observed 809.

### Example 43

### [(4S,5R)-2-[4-Chloro-2-(2-fluoro-ethoxy)-5-methanesulfonyl-phenyl]-4,5-bis-(4-chloro-phenyl)-4,5-dimethyl-4,5-dihydro-imidazol-1-yl]-[4-(3-methanesulfonyl-propyl)-piperazin-1-yl]-methanone

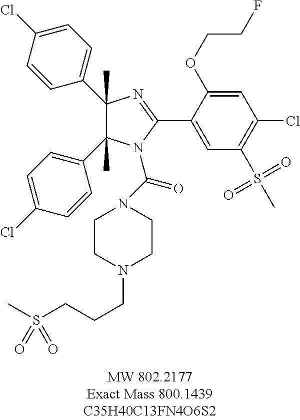 [SMILES: C[C@@]1(c2ccc(Cl)cc2)N=C(c2cc(S(C)(=O)=O)c(Cl)cc2OCCF)N(C(=O)N2CCN(CCCS(C)(=O)=O)CC2)[C@]1(C)c1ccc(Cl)cc1]

In a manner analogous to the method described in Example 40, rac-[(4S,5R)-2-(4-chloro-2-hydroxy-5-methanesulfonyl-phenyl)-4,5-bis-(4-chloro-phenyl)-4,5-dimethyl-4,5-dihydro-imidazol-1-yl]-[4-(3-methanesulfonyl-propyl)-piperazin-1-yl]-methanone was alkylated with 1-fluoro-2-iodoethane to give the title compound. LC-MS (ES, m/z) calculated for C₃₅H₄₁Cl₃FN₄O₆S₂ [(M+H)⁺] 801, observed 801.

### Example 44

### [(4S,5R)-2-(2-sec-Butoxy-4-chloro-5-methanesulfonyl-phenyl)-4,5-bis-(4-chloro-phenyl)-4,5-dimethyl-4,5-dihydro-imidazol-1-yl]-[4-(3-methanesulfonyl-propyl)-piperazin-1-yl]-methanone

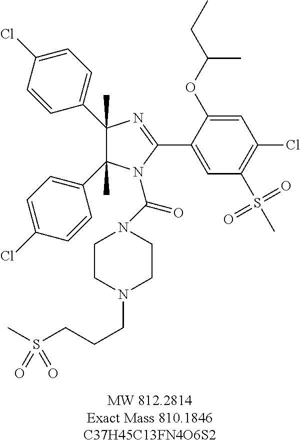 [SMILES: CCC(C)Oc1cc(Cl)c(S(C)(=O)=O)cc1C1=N[C@@](C)(c2ccc(Cl)cc2)[C@@](C)(c2ccc(Cl)cc2)N1C(=O)N1CCN(CCCS(C)(=O)=O)CC1]

In a manner analogous to the method described in Example 40, rac-[(4S,5R)-2-(4-chloro-2-hydroxy-5-methanesulfonyl-phenyl)-4,5-bis-(4-chloro-phenyl)-4,5-dimethyl-4,5-dihydro-imidazol-1-yl]-[4-(3-methanesulfonyl-propyl)-piperazin-1-yl]-methanone was alkylated with 2-iodobutane to give the title compound. LC-MS (ES, m/z) calculated for C₃₇H₄₆Cl₃N₄O₆S₂ [(M+H)⁺] 811, observed 811.

### Example 45

### 5-{(4S,5R)-4,5-Bis-(4-chloro-phenyl)-1-[4-(3,4-dihydroxy-butyl)-piperazine-1-carbonyl]-4,5-dimethyl-4,5-dihydro-1H-imidazol-2-yl}-N-tert-butyl-2-chloro-4-ethoxy-benzene sulfonamide

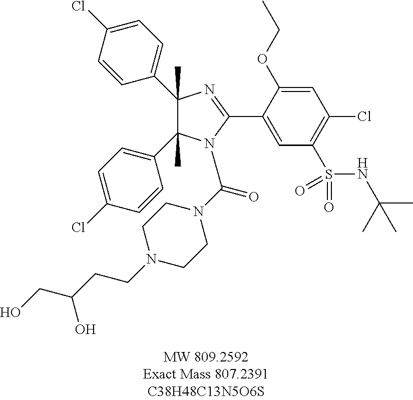 [SMILES: CCOc1cc(Cl)c(S(=O)(=O)NC(C)(C)C)cc1C1=N[C@@](C)(c2ccc(Cl)cc2)[C@@](C)(c2ccc(Cl)cc2)N1C(=O)N1CCN(CCC(O)CO)CC1]

4-Chlorosalicylic acid (15 g, Aldrich, 87.5 mmol) and ethyl iodide (35.7 g, 230 mmol) were dissolved in dimethylformamide (400 mL) and the solution was cooled to 0° C. (acetone-ice bath) with good paddle stirring. Sodium hydride (10.5 g, Aldrich, 50% in oil, 220 mmol) was added in two lots. Cooling was removed after 10 min. and the reaction warmed slowly to 30° C. at which point heating was increased to 50-60° C. for 2 h. The reaction was cooled and poured into ice water and extracted into dichloromethane (3×500 mL). The organic layer was washed with water (2×500 mL), washed with brine, dried over magnesium sulfate, filtered and evaporated to dryness to give 4-chloro-2-ethoxybenzoic acid ethyl ester.

4-Chloro-2-ethoxybenzoic acid ethyl ester (˜18 g) was suspended in ethanol (500 mL). This was treated with potassium hydroxide (200 mL, 2 M solution) and refluxed for 1 h. The reaction was cooled and poured into cold aqueous hydrochloric acid (300 mL, 3 M solution) and extracted into dichloromethane (3×500 mL). The dichloromethane was washed with water (500 mL), dried over magnesium sulfate, filtered and evaporated to dryness to give 4-chloro-2-ethoxybenzoic acid as a solid.

4-Chloro-2-ethoxybenzoic acid (9.4 g, 47 mmol) was added in several portions to cold chlorosulfonic acid (55 mL, Aldrich) stirring at 0-10° C., and the resulting solution was heated at ˜60° C. for 2 h. The resulting dark solution was poured into ice (800 g), and then 500 mL of dichloromethane was added. After stirring for 15 min, the layers were separated and the aqueous layer was extracted with 200 mL of dichloromethane. The organic layers were washed in turn with brine, combined, dried over magnesium sulfate, filtered and concentrated at room temperature. Crystallization from ether/hexane gave 3.4 g of 4-chloro-5-chlorosulfonyl-2-ethoxybenzoic acid.

A solution of 4-chloro-6-chlorosulfonyl-2-ethoxybenzoic acid (1.0 g, 3.34 mmol) and tert-butyl amine (2.0 mL) in 20 mL of tetrahydrofuran was refluxed for 4 h and concentrated. The residue in water was acidified and extracted with ether, the extract was washed with water, brine, dried over magnesium sulfate, filtered and evaporated. Precipitation from ether/hexane provided 0.8 g of crude 5-tert-butylsulfamoyl-4-chloro-2-ethoxybenzoic acid which was used without further purification.

5-tert-Butylsulfamoyl-4-chloro-2-ethoxybenzoic acid (0.8 g, 24 mmol) in 10 mL of tetrahydrofuran was treated with diazomethane (15 mL, ˜2-3 M in ether, prepared from N-methyl-N-nitroso-p-toluenesulfonamide, Aldrich) at room temperature and stirred for 0.5 h. The mixture was concentrated, and the residue was purified by flash chromatography (silica gel, eluting with 20% ethyl acetate in hexane) to give 0.45 g of 5-tert-butylsulfamoyl-4-chloro-2-ethoxybenzoic acid methyl ester as a solid.

In a manner analogous to the method described in Example 55, meso-2,3-bis-(4-chlorophenyl)-2,3-butanediamine (prepared as described in Ding et al. U.S. Pat. No. 7,851,626) was reacted with 5-tert-butylsulfamoyl-4-chloro-2-ethoxybenzoic acid methyl ester in the presence of trimethylaluminum to give rac-5-[(4S,5R)-4,5-bis-(4-chloro-phenyl)-4,5-dimethyl-4,5-dihydro-1H-imidazol-2-yl]-N-tert-butyl-2-chloro-4-ethoxy-benzenesulfonamide. HR-MS (ES, m/z) calculated for C₂₉H₃₃N₃O₃SCl₃ [(M+H)⁺] 608.1303, observed 608.1300.

In a manner analogous to the method described in Example 1, rac-5-[(4S,5R)-4,5-bis-(4-chloro-phenyl)-4,5-dimethyl-4,5-dihydro-1H-imidazol-2-yl]-N-tert-butyl-2-chloro-4-ethoxy-benzenesulfonamide was reacted with phosgene in the presence of triethylamine to give rac-(4S,5R)-2-(5-tert-butylsulfamoyl-4-chloro-2-ethoxy-phenyl)-4,5-bis-(4-chloro-phenyl)-4,5-dimethyl-4,5-dihydro-imidazole-1-carbonyl chloride.

In a manner analogous to the method described in Example 2, rac-(4S,5R)-2-(5-tert-butylsulfamoyl-4-chloro-2-ethoxy-phenyl)-4,5-bis-(4-chloro-phenyl)-4,5-dimethyl-4,5-dihydro-imidazole-1-carbonyl chloride was reacted with 4-(3,4-dihydroxy-butyl)-piperazine to give the tittle compound. HR-MS (ES, m/z) calculated for C₃₈H₄₉Cl₃N₅O₆S [(M+H)⁺] 808.2464, observed 808.2469.

### Example 46

### 5-((4S,5R)-4,5-Bis-(4-chloro-phenyl)-1-{4-[2-(2-hydroxy-ethoxy)-ethyl]-piperazine-1-carbonyl}-4,5-dimethyl-4,5-dihydro-1H-imidazol-2-yl)-N-tert-butyl-2-chloro-4-ethoxy-benzenesulfonamide

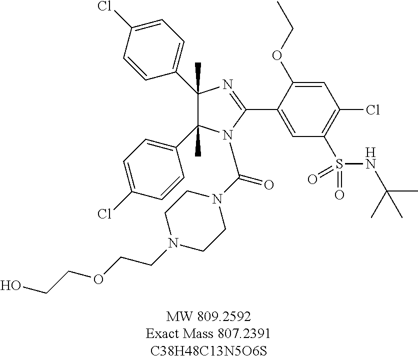 [SMILES: CCOc1cc(Cl)c(S(=O)(=O)NC(C)(C)C)cc1C1=N[C@@](C)(c2ccc(Cl)cc2)[C@@](C)(c2ccc(Cl)cc2)N1C(=O)N1CCN(CCOCCO)CC1]

In a manner analogous to the method described in Example 45, rac-(4S,5R)-2-(5-tert-butylsulfamoyl-4-chloro-2-ethoxy-phenyl)-4,5-bis-(4-chloro-phenyl)-4,5-dimethyl-4,5-dihydro-imidazole-1-carbonyl chloride was reacted with 4-[2-(2-hydroxy-ethoxy)-ethyl]-piperazine to give the tittle compound. HR-MS (ES, m/z) calculated for C₃₈H₄₉Cl₃N₅O₆S [(M+H)⁺] 808.2464, observed 808.2467.

### Example 47

### 5-((4S,5R)-4,5-Bis-(4-chloro-phenyl)-4,5-dimethyl-1-{4-[2-(4-methyl-piperazin-1-yl)-2-oxo-ethyl]-piperazine-1-carbonyl}-4,5-dihydro-1H-imidazol-2-yl)-N-tert-butyl-2-chloro-4-ethoxy-benzenesulfonamide

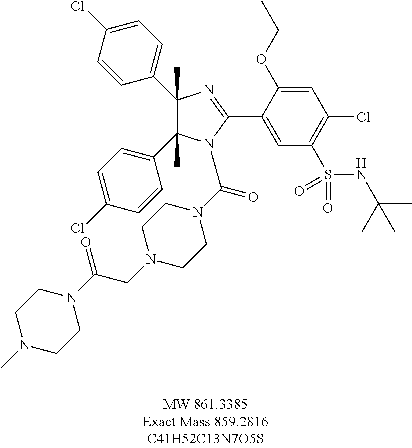 [SMILES: CCOc1cc(Cl)c(S(=O)(=O)NC(C)(C)C)cc1C1=N[C@@](C)(c2ccc(Cl)cc2)[C@@](C)(c2ccc(Cl)cc2)N1C(=O)N1CCN(CC(=O)N2CCN(C)CC2)CC1]

In a manner analogous to the method described in Example 45, rac-(4S,5R)-2-(5-tert-butylsulfamoyl-4-chloro-2-ethoxy-phenyl)-4,5-bis-(4-chloro-phenyl)-4,5-dimethyl-4,5-dihydro-imidazole-1-carbonyl chloride was reacted with 4-[2-(4-methyl-piperazin-1-yl)-2-oxo-ethyl]-piperazine to give the tittle compound. HR-MS (ES, m/z) calculated for C₄₁H₅₃Cl₃N₇O₅S [(M+H)⁺] 860.2889, observed 860.2889.

### Example 48

### 2-{4-[(4S,5R)-2-(5-tert-Butylsulfamoyl-4-chloro-2-ethoxy-phenyl)-4,5-bis-(4-chloro-phenyl)-4,5-dimethyl-4,5-dihydro-imidazole-1-carbonyl]-piperazin-1-yl}-N-(tetrahydro-furan-2-ylmethyl)-acetamide

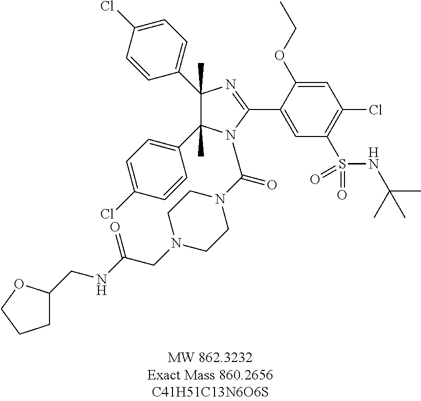 [SMILES: CCOc1cc(Cl)c(S(=O)(=O)NC(C)(C)C)cc1C1=N[C@@](C)(c2ccc(Cl)cc2)[C@@](C)(c2ccc(Cl)cc2)N1C(=O)N1CCN(CC(=O)NCC2CCCO2)CC1]

In a manner analogous to the method described in Example 45, rac-(4S,5R)-2-(5-tert-butylsulfamoyl-4-chloro-2-ethoxy-phenyl)-4,5-bis-(4-chloro-phenyl)-4,5-dimethyl-4,5-dihydro-imidazole-1-carbonyl chloride was reacted with 2-piperazin-1-yl-N-(tetrahydro-furan-2-ylmethyl)-acetamide to give the tittle compound. HR-MS (ES, m/z) calculated for C₄₁H₅₂Cl₃N₆O₆S [(M+H)⁺] 861.2729, observed 861.2730.

### Example 49

### 2-{4-[(4S,5R)-2-(5-tert-Butylsulfamoyl-4-chloro-2-ethoxy-phenyl)-4,5-bis-(4-chloro-phenyl)-4,5-dimethyl-4,5-dihydro-imidazole-1-carbonyl]-piperazin-1-yl}-N-(2-methoxy-ethyl)-acetamide

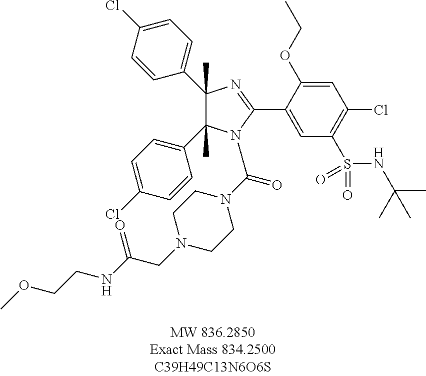 [SMILES: CCOc1cc(Cl)c(S(=O)(=O)NC(C)(C)C)cc1C1=N[C@@](C)(c2ccc(Cl)cc2)[C@@](C)(c2ccc(Cl)cc2)N1C(=O)N1CCN(CC(=O)NCCOC)CC1]

In a manner analogous to the method described in Example 45, rac-(4S,5R)-2-(5-tert-butylsulfamoyl-4-chloro-2-ethoxy-phenyl)-4,5-bis-(4-chloro-phenyl)-4,5-dimethyl-4,5-dihydro-imidazole-1-carbonyl chloride was reacted with N-(2-methoxy-ethyl)-piperazin-1-yl-acetamide to give the tittle compound. HR-MS (ES, m/z) calculated for C₃₉H₅₀Cl₃N₆O₆S [(M+H)⁺] 835.2573, observed 835.2575.

### Example 50

### 3-{4-[(4S,5R)-2-(5-tert-Butylsulfamoyl-4-chloro-2-ethoxy-phenyl)-4,5-bis-(4-chloro-phenyl)-4,5-dimethyl-4,5-dihydro-imidazole-1-carbonyl]-piperazin-1-yl}-propionamide

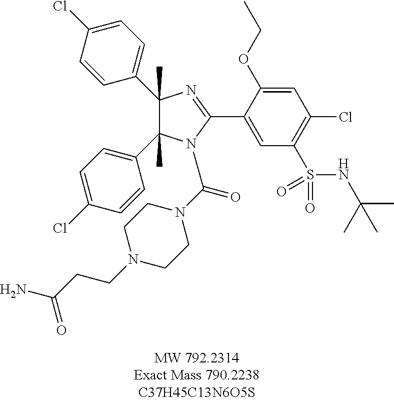 [SMILES: CCOc1cc(Cl)c(S(=O)(=O)NC(C)(C)C)cc1C1=N[C@@](C)(c2ccc(Cl)cc2)[C@@](C)(c2ccc(Cl)cc2)N1C(=O)N1CCN(CCC(N)=O)CC1]

In a manner analogous to the method described in Example 45, rac-(4S,5R)-2-(5-tert-butylsulfamoyl-4-chloro-2-ethoxy-phenyl)-4,5-bis-(4-chloro-phenyl)-4,5-dimethyl-4,5-dihydro-imidazole-1-carbonyl chloride was reacted with piperazin-1-yl-propionamide to give the tittle compound. HR-MS (ES, m/z) calculated for C₃₇H₄₆Cl₃N₆O₅S [(M+H)⁺] 791.2311, observed 791.2312.

### Example 51

### 5-{(4S,5R)-4,5-Bis-(4-chloro-phenyl)-1-[4-(1,1-dioxo-tetrahydro-thiophen-3-yl)-piperazine-1-carbonyl]-4,5-dimethyl-4,5-dihydro-1H-imidazol-2-yl}-N-tert-butyl-2-chloro-4-ethoxy-benzenesulfonamide

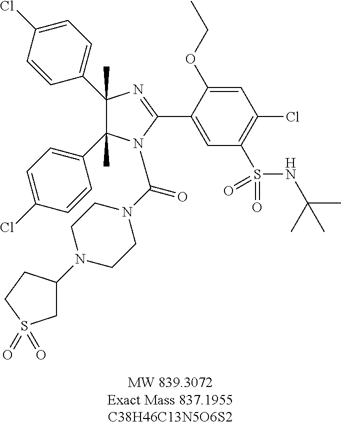 [SMILES: CCOc1cc(Cl)c(S(=O)(=O)NC(C)(C)C)cc1C1=N[C@@](C)(c2ccc(Cl)cc2)[C@@](C)(c2ccc(Cl)cc2)N1C(=O)N1CCN(C2CCS(=O)(=O)C2)CC1]

In a manner analogous to the method described in Example 45, rac-(4S,5R)-2-(5-tert-butylsulfamoyl-4-chloro-2-ethoxy-phenyl)-4,5-bis-(4-chloro-phenyl)-4,5-dimethyl-4,5-dihydro-imidazole-1-carbonyl chloride was reacted with 4-(1,1-dioxo-tetrahydro-thiophen-3-yl)-piperazine to give the tittle compound. HR-MS (ES, m/z) calculated for C₃₈H₄₇Cl₃N₅O₆S₂ [(M+H)⁺] 838.2028, observed 838.2032.

### Example 52

### 5-{(4S,5R)-4,5-Bis-(4-chloro-phenyl)-4,5-dimethyl-1-[4-(2-tetrazol-1-yl-acetyl)-piperazine-1-carbonyl]-4,5-dihydro-1H-imidazol-2-yl}-N-tert-butyl-2-chloro-4-ethoxy-benzene sulfonamide

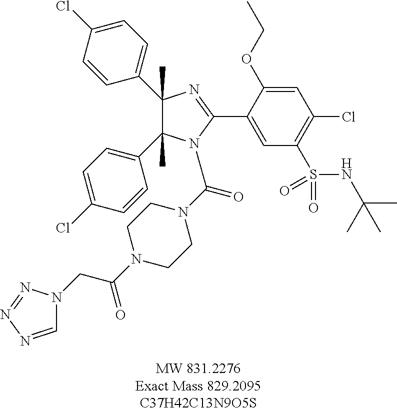 [SMILES: CCOc1cc(Cl)c(S(=O)(=O)NC(C)(C)C)cc1C1=N[C@@](C)(c2ccc(Cl)cc2)[C@@](C)(c2ccc(Cl)cc2)N1C(=O)N1CCN(C(=O)Cn2cnnn2)CC1]

In a manner analogous to the method described in Example 45, rac-(4S,5R)-2-(5-tert-butylsulfamoyl-4-chloro-2-ethoxy-phenyl)-4,5-bis-(4-chloro-phenyl)-4,5-dimethyl-4,5-dihydro-imidazole-1-carbonyl chloride was reacted with 4-(2-tetrazol-1-yl-acetyl)-piperazine to give the tittle compound. HR-MS (ES, m/z) calculated for C₃₇H₄₃Cl₃N₉O₅S [(M+H)⁺] 830.2168, observed 830.2165.

### Example 53

### 5-{(4S,5R)-4,5-Bis-(4-chloro-phenyl)-4,5-dimethyl-1-[4-(5-oxo-[1,4]diazepan-1-yl)-piperidine-1-carbonyl]-4,5-dihydro-1H-imidazol-2-yl}-N-tert-butyl-2-chloro-4-ethoxy-benzene sulfonamide

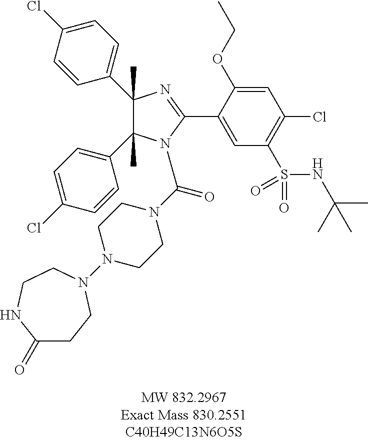 [SMILES: CCOc1cc(Cl)c(S(=O)(=O)NC(C)(C)C)cc1C1=N[C@@](C)(c2ccc(Cl)cc2)[C@@](C)(c2ccc(Cl)cc2)N1C(=O)N1CCN(N2CCNC(=O)CC2)CC1]

In a manner analogous to the method described in Example 45, rac-(4S,5R)-2-(5-tert-butylsulfamoyl-4-chloro-2-ethoxy-phenyl)-4,5-bis-(4-chloro-phenyl)-4,5-dimethyl-4,5-dihydro-imidazole-1-carbonyl chloride was reacted with 4-(5-oxo-[1,4]diazepan-1-yl)-piperidine to give the tittle compound. HR-MS (ES, m/z) calculated for C₄₀H₅₀Cl₃N₆O₅S [(M+H)⁺] 831.2624, observed 831.2622.

### Example 54

### 5-[(4S,5R)-4,5-Bis-(4-chloro-phenyl)-1-(3-hydroxy-piperidine-1-carbonyl)-4,5-dimethyl-4,5-dihydro-1H-imidazol-2-yl]-N-tert-butyl-2-chloro-4-ethoxy-benzenesulfonamide

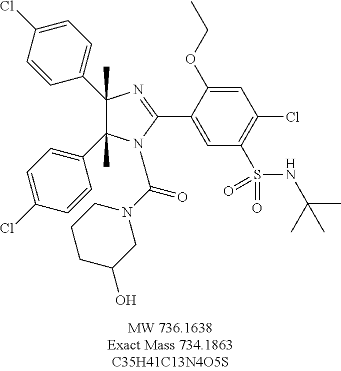 [SMILES: CCOc1cc(Cl)c(S(=O)(=O)NC(C)(C)C)cc1C1=N[C@@](C)(c2ccc(Cl)cc2)[C@@](C)(c2ccc(Cl)cc2)N1C(=O)N1CCCC(O)C1]

In a manner analogous to the method described in Example 45, rac-(4S,5R)-2-(5-tert-butylsulfamoyl-4-chloro-2-ethoxy-phenyl)-4,5-bis-(4-chloro-phenyl)-4,5-dimethyl-4,5-dihydro-imidazole-1-carbonyl chloride was reacted with 3-hydroxy-piperidine to give the tittle compound. HR-MS (ES, m/z) calculated for C₃₅H₄₂Cl₃N₄O₅S [(M+H)⁺] 735.1936, observed 735.1934.

### Example 55

### {(4S,5R)-4,5-Bis-(4-chloro-phenyl)-2-[4-chloro-2-propoxy-5-(pyrrolidine-1-sulfonyl)-phenyl]-4,5-dimethyl-4,5-dihydro-imidazol-1-yl}-[4-(3-methanesulfonyl-propyl)-piperazin-1-yl]-methanone

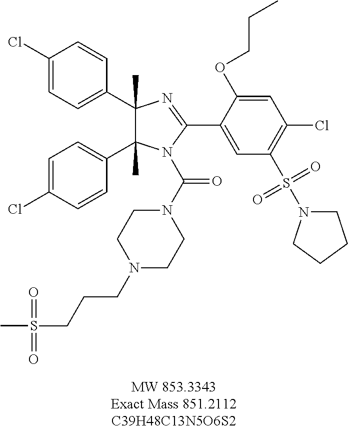 [SMILES: CCCOc1cc(Cl)c(S(=O)(=O)N2CCCC2)cc1C1=N[C@@](C)(c2ccc(Cl)cc2)[C@@](C)(c2ccc(Cl)cc2)N1C(=O)N1CCN(CCCS(C)(=O)=O)CC1]

To a solution of 4-chloro-5-chlorosulfonyl-2-hydroxy-benzoic acid (6 g, 22 mmol, example 1) in anhydrous tetrahydrofuran (50 mL) cooled to 0° C. was added pyrrolidine (10 eq) dropwise. After 10 min, the reaction mixture was poured into iced water and acidified with cold 1N hydrochloric acid. The product was extracted with ethyl acetate. The organic layer was washed with water, brine, dried over anhydrous sodium sulfate, filtered and concentrated to give crude 4-chloro-2-hydroxy-5-(pyrrolidine-1-sulfonyl)-benzoic acid as white solids (8 g). It was used without further purification.

The mixture of 4-chloro-2-hydroxy-5-(pyrrolidine-1-sulfonyl)-benzoic acid (500 mg, 1.64 mmol), potassium carbonate (793 mg, 3.5 mmol), propyl iodide (640 uL, 6.56 mmol) in dimethylformamide (4 mL) was heated at 100° C. overnight. Upon cooling to room temperature, the reaction mixture was concentrated, and water was added. The product was extracted with ethyl acetate (2×). The organic extracts were washed with water, brine, dried over anhydrous sodium sulfate, filtered and concentrated. Purification of the crude residue by flash column chromatography (40 g of silica gel, eluting with a gradient of ethyl acetate in hexanes) gave propyl 4-chloro-2-propoxy-5-(pyrrolidine-1-sulfonyl)-benzoate (537 mg).

meso-2,3-Bis-(4-chlorophenyl)-2,3-butanediamine (424 mg, 1.37 mmol, prepared as described in Ding et al. U.S. Pat. No. 7,851,626) was added to a solution of trimethylaluminum (0.68 mL, 1M in toluene) in toluene (15 mL) cooled to 0° C. After stirring at 0° C. for 10 min and at room temperature for 15 min, the mixture was heated at 60° C. for 20 min, at 90° C. for 30 min (gas bubbles seen). Upon cooling to room temperature, propyl 4-chloro-2-propoxy-5-(pyrrolidine-1-sulfonyl)-benzoate (534 mg, 1.37 mmol) was added and the mixture was heated at reflux overnight. Upon cooling to room temperature, 1M Rochelle salt solution was added, and the biphasic mixture was stirred vigorously at room temperature overnight. The layers were separated, and the product was extracted with ethyl acetate. The organic extracts were washed with water, brine, dried over anhydrous sodium sulfate, filtered and concentrated to give rac-(4S,5R)-4,5-bis-(4-chloro-phenyl)-2-[4-chloro-2-propoxy-5-(pyrrolidine-1-sulfonyl)-phenyl]-4,5-dimethyl-4,5-dihydro-1H-imidazole (920 mg). It was converted to the corresponding carbonyl chloride as described in example 1 without further purification.

In a manner analogous to the method described in Example 2, rac-(4S,5R)-4,5-bis-(4-chloro-phenyl)-2-[4-chloro-2-propoxy-5-(pyrrolidine-1-sulfonyl)-phenyl]-4,5-dimethyl-4,5-dihydro-1H-imidazole-1-carbonyl chloride was reacted with 4-(3-methanesulfonyl-propyl)-piperazine (prepared as described in Ding et al. U.S. Pat. No. 7,851,626) to give the tittle compound. HR-MS (ES, m/z) calculated for C₃₉H₄₉Cl₃N₅O₆S₂ [(M+H)⁺] 852.2185, observed 852.2179.

### Example 56

### 2-{1-[(4S,5R)-2-[4-Chloro-2-ethoxy-5-(pyrrolidine-1-sulfonyl)-phenyl]-4,5-bis-(4-chloro-phenyl)-4,5-dimethyl-4,5-dihydro-imidazole-1-carbonyl]-piperidin-4-yl}-acetamide

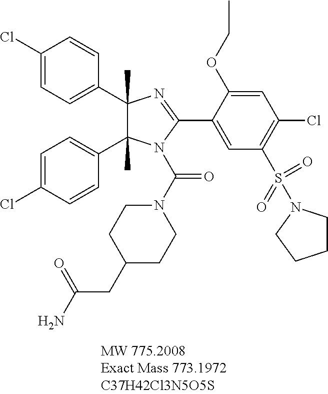 [SMILES: CCOc1cc(Cl)c(S(=O)(=O)N2CCCC2)cc1C1=N[C@@](C)(c2ccc(Cl)cc2)[C@@](C)(c2ccc(Cl)cc2)N1C(=O)N1CCC(CC(N)=O)CC1]

The mixture of 4-chloro-2-hydroxy-5-(pyrrolidine-1-sulfonyl)-benzoic acid (8 g, prepared as described in example 55), potassium carbonate (3.5 eq.), ethyl iodide (4 eq.) in dimethylformamide (65 mL) was heated at 100° C. for 2 h. Upon cooling to room temperature, the reaction mixture was concentrated, and water was added. The product was extracted with ethyl acetate (2×). The organic extracts were washed with water, brine, dried over anhydrous sodium sulfate, filtered and concentrated. Purification of the crude residue by flash column chromatography (330 g of silica gel, eluting with a gradient of ethyl acetate in hexanes) gave ethyl 4-chloro-2-ethoxy-5-(pyrrolidine-1-sulfonyl)-benzoate (7.34 g).

meso-2,3-Bis-(4-chlorophenyl)-2,3-butanediamine (500 mg, 1.62 mmol, prepared as described in Ding et al. U.S. Pat. No. 7,851,626) was added to a solution of trimethylaluminum (0.89 mL, 1M in toluene) in toluene (15 mL) cooled to 0° C. After stirring at 0° C. for 10 min and at room temperature for 15 min, the mixture was heated at 60° C. for 20 min, at 90° C. for 30 min (gas bubbles seen). Upon cooling to room temperature, a solution of ethyl 4-chloro-2-ethoxy-5-(pyrrolidine-1-sulfonyl)-benzoate (586 mg, 1.62 mmol) in 5 mL of toluene was added, and the mixture was heated at reflux overnight. Upon cooling to room temperature, 1M Rochelle salt solution was added, and the biphasic mixture was stirred vigorously at room temperature overnight. The layers were separated, and the product was extracted with ethyl acetate. The organic extracts were washed with water, brine, dried over anhydrous sodium sulfate, filtered and concentrated. Purification of the crude residue by flash column chromatography (40 g of silica gel, eluting with a gradient of ethyl acetate in hexanes) rac-(4S,5R)-4,5-bis-(4-chloro-phenyl)-2-[4-chloro-2-ethoxy-5-(pyrrolidine-1-sulfonyl)-phenyl]-4,5-dimethyl-4,5-dihydro-1H-imidazole (790 mg). It was converted to the corresponding carbonyl chloride as described in Example 1.

In a manner analogous to the method described in Example 2, rac-(4S,5R)-2-[4-chloro-2-ethoxy-5-(pyrrolidine-1-sulfonyl)-phenyl]-4,5-bis-(4-chloro-phenyl)-4,5-dimethyl-4,5-dihydro-1H-imidazole-1-carbonyl chloride was reacted with piperidine-4-yl-acetamide to give the tittle compound. HR-MS (ES, m/z) calculated for C₃₇H₄₃Cl₃N₅O₅S [(M+H)⁺] 774.2045, observed 774.2051.

### Example 57

### [(4S,5R)-2-[4-Chloro-2-ethoxy-5-(morpholine-4-sulfonyl)-phenyl]-4,5-bis-(4-chloro-phenyl)-4,5-dimethyl-4,5-dihydro-imidazol-1-yl]-[4-(3-methanesulfonyl-propyl)-piperazin-1-yl]-methanone

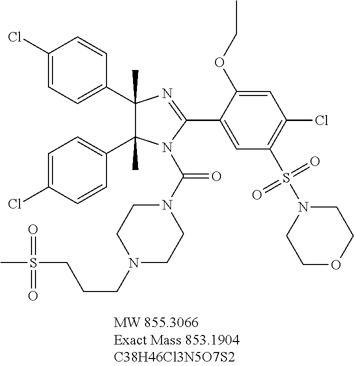 [SMILES: CCOc1cc(Cl)c(S(=O)(=O)N2CCOCC2)cc1C1=N[C@@](C)(c2ccc(Cl)cc2)[C@@](C)(c2ccc(Cl)cc2)N1C(=O)N1CCN(CCCS(C)(=O)=O)CC1]

In a manner analogous to the method described in Example 2, rac-(4S,5R)-2-[4-chloro-2-ethoxy-5-(morpholine-4-sulfonyl)-phenyl]-4,5-bis-(4-chloro-phenyl)-4,5-dimethyl-4,5-dihydro-imidazole-1-carbonyl chloride (prepared in an analogous manner as described in example 55) was reacted with 4-(3-methanesulfonyl-propyl)-piperazine (prepared as described in Ding et al. U.S. Pat. No. 7,851,626) to give the tittle compound. HR-MS (ES, m/z) calculated for C₃₈H₄₇Cl₃N₅O₇S₂ [(M+H)⁺] 854.1977, observed 854.1980.

### Example 58

### [(4S,5R)-2-[4-Chloro-2-isopropoxy-5-(pyrrolidine-1-sulfonyl)-phenyl]-4,5-bis-(4-chloro-phenyl)-4,5-dimethyl-4,5-dihydro-imidazol-1-yl]-[4-(3-methanesulfonyl-propyl)-piperazin-1-yl]-methanone

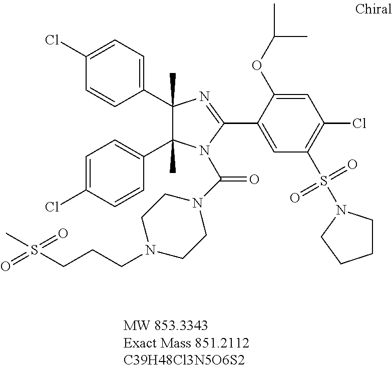 [SMILES: CC(C)Oc1cc(Cl)c(S(=O)(=O)N2CCCC2)cc1C1=N[C@@](C)(c2ccc(Cl)cc2)[C@@](C)(c2ccc(Cl)cc2)N1C(=O)N1CCN(CCCS(C)(=O)=O)CC1]

In a manner analogous to the method described in Example 2, rac-(4S,5R)-2-[4-chloro-2-isopropoxy-5-(pyrrolidine-1-sulfonyl)-phenyl]-4,5-bis-(4-chloro-phenyl)-4,5-dimethyl-4,5-dihydro-1H-imidazole-1-carbonyl chloride (prepared in an analogous manner as described in example 55) was reacted with 4-(3-methanesulfonyl-propyl)-piperazine (prepared as described in Ding et al. U.S. Pat. No. 7,851,626) to give the tittle compound. HR-MS (ES, m/z) calculated for C₃₉H₄₉Cl₃N₅O₆S₂ [(M+H)⁺] 852.2185, observed 852.2185.

### Example 59

### 2-{1-[(4S,5R)-2-[4-Chloro-2-isopropoxy-5-(pyrrolidine-1-sulfonyl)-phenyl]-4,5-bis-(4-chloro-phenyl)-4,5-dimethyl-4,5-dihydro-imidazole-1-carbonyl]-piperidin-4-yl}-acetamide

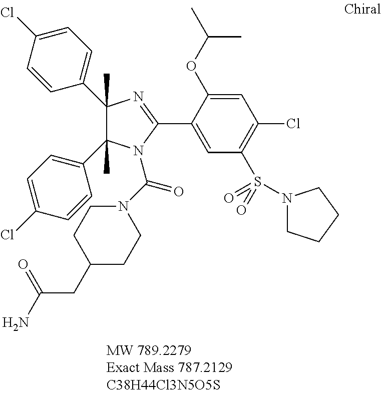 [SMILES: CC(C)Oc1cc(Cl)c(S(=O)(=O)N2CCCC2)cc1C1=N[C@@](C)(c2ccc(Cl)cc2)[C@@](C)(c2ccc(Cl)cc2)N1C(=O)N1CCC(CC(N)=O)CC1]

In a manner analogous to the method described in Example 2, rac-(4S,5R)-2-[4-chloro-2-isopropoxy-5-(pyrrolidine-1-sulfonyl)-phenyl]-4,5-bis-(4-chloro-phenyl)-4,5-dimethyl-4,5-dihydro-1H-imidazole-1-carbonyl chloride (prepared in an analogous manner as described in Example 55) was reacted with piperidine-4-yl-acetamide to give the tittle compound. LC-MS (ES, m/z) calculated for C₃₈H₄₅Cl₃N₅O₅S [(M+H)⁺] 788, observed 788.

### Example 60

### 2-{1-[(4S,5R)-2-(5-tert-Butylsulfamoyl-4-chloro-2-isopropoxy-phenyl)-4,5-bis-(4-chloro-phenyl)-4,5-dimethyl-4,5-dihydro-imidazole-1-carbonyl]-piperidin-4-yl}-acetamide

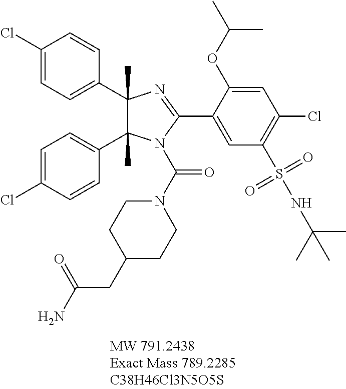 [SMILES: CC(C)Oc1cc(Cl)c(S(=O)(=O)NC(C)(C)C)cc1C1=N[C@@](C)(c2ccc(Cl)cc2)[C@@](C)(c2ccc(Cl)cc2)N1C(=O)N1CCC(CC(N)=O)CC1]

In a manner analogous to the method described in Example 2, rac-(4S,5R)-2-(5-tert-butylsulfamoyl-4-chloro-2-isopropoxy-phenyl)-4,5-bis-(4-chloro-phenyl)-4,5-dimethyl-4,5-dihydro-imidazole-1-carbonyl chloride (prepared as described in Example 45) was reacted with piperidine-4-yl-acetamide to give the tittle compound. LC-MS (ES, m/z) calculated for C₃₈H₄₇Cl₃N₅O₅S [(M+H)⁺] 790, observed 790.

### Example 61

### 2-{1-[(4S,5R)-2-{4-Chloro-2-isopropoxy-5-[(1R,5S)-(8-oxa-3-aza-bicyclo[3.2.1]oct-3-yl)sulfonyl]-phenyl}-4,5-bis-(4-chloro-phenyl)-4,5-dimethyl-4,5-dihydro-imidazole-1-carbonyl]-piperidin-4-yl}-acetamide

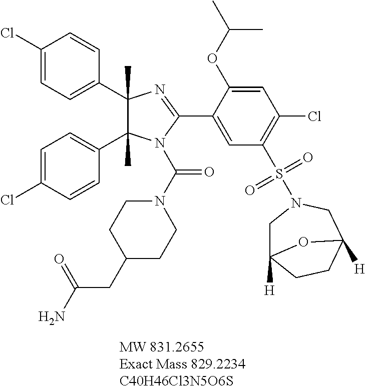 [SMILES: CC(C)Oc1cc(Cl)c(S(=O)(=O)N2C[C@H]3CC[C@@H](C2)O3)cc1C1=N[C@@](C)(c2ccc(Cl)cc2)[C@@](C)(c2ccc(Cl)cc2)N1C(=O)N1CCC(CC(N)=O)CC1]

In a manner analogous to the method described in Example 2, rac-(4S,5R)-2-{4-chloro-2-isopropoxy-5-[(1R,5S)-(8-oxa-3-aza-bicyclo[3.2.1]oct-3-yl)sulfonyl]-phenyl}-4,5-bis-(4-chloro-phenyl)-4,5-dimethyl-4,5-dihydro-imidazole-1-carbonyl chloride (prepared in an analogous manner as described in Example 55) was reacted with piperidine-4-yl-acetamide to give the tittle compound. LC-MS (ES, m/z) calculated for C₄₀H₄₇Cl₃N₅O₆S [(M+H)⁺] 830, observed 830.

### Example 62

### [(4S,5R)-2-{4-Chloro-2-isopropoxy-5-[(1R,5S)-(8-oxa-3-aza-bicyclo[3.2.1]oct-3-yl)sulfonyl]-phenyl}-4,5-bis-(4-chloro-phenyl)-4,5-dimethyl-4,5-dihydro-imidazol-1-yl]-[4-(3-methanesulfonyl-propyl)-piperazin-1-yl]-methanone

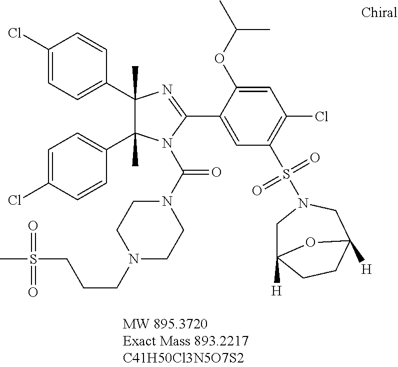 [SMILES: CC(C)Oc1cc(Cl)c(S(=O)(=O)N2C[C@H]3CC[C@@H](C2)O3)cc1C1=N[C@@](C)(c2ccc(Cl)cc2)[C@@](C)(c2ccc(Cl)cc2)N1C(=O)N1CCN(CCCS(C)(=O)=O)CC1]

In a manner analogous to the method described in Example 2, rac-(4S,5R)-2-{4-chloro-2-isopropoxy-5-[(1R,5S)-(8-oxa-3-aza-bicyclo[3.2.1]oct-3-yl)sulfonyl]-phenyl}-4,5-bis-(4-chloro-phenyl)-4,5-dimethyl-4,5-dihydro-imidazole-1-carbonyl chloride (prepared in an analogous manner as described in Example 55) was reacted with 4-(3-methanesulfonyl-propyl)-piperazine (prepared as described in Ding et al. U.S. Pat. No. 7,851,626) to give the tittle compound. LC-MS (ES, m/z) calculated for C₄₁H₅₁Cl₃N₅O₇S₂ [(M+H)⁺] 894, observed 894.

### Example 63

### 5-{(4S,5R)-4,5-Bis-(4-chloro-phenyl)-1-[4-(3-methanesulfonyl-propyl)-piperazine-1-carbonyl]-4,5-dimethyl-4,5-dihydro-1H-imidazol-2-yl}-N-tert-butyl-2-chloro-4-isopropoxy-benzenesulfonamide

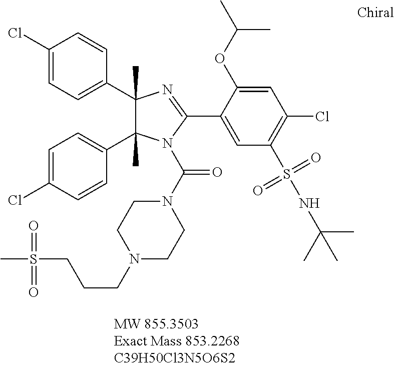 [SMILES: CC(C)Oc1cc(Cl)c(S(=O)(=O)NC(C)(C)C)cc1C1=N[C@@](C)(c2ccc(Cl)cc2)[C@@](C)(c2ccc(Cl)cc2)N1C(=O)N1CCN(CCCS(C)(=O)=O)CC1]

In a manner analogous to the method described in Example 45, rac-(4S,5R)-2-(5-tert-butylsulfamoyl-4-chloro-2-isopropoxy-phenyl)-4,5-bis-(4-chloro-phenyl)-4,5-dimethyl-4,5-dihydro-imidazole-1-carbonyl chloride was reacted with 4-(3-methanesulfonyl-propyl)-piperazine (prepared as described in Ding et al. U.S. Pat. No. 7,851,626) to give the tittle compound. LC-MS (ES, m/z) calculated for C₃₉H₅₁Cl₃N₅O₆S₂ [(M+H)⁺] 854, observed 854.

### Example 64

### 2-{1-[(4S,5R)-2-(4-Chloro-2-isopropoxy-5-sulfamoyl-phenyl)-4,5-bis-(4-chloro-phenyl)-4,5-dimethyl-4,5-dihydro-imidazole-1-carbonyl]-piperidin-4-yl}-acetamide

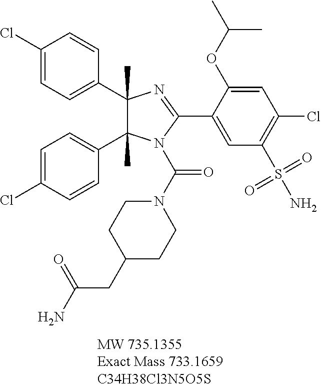 [SMILES: CC(C)Oc1cc(Cl)c(S(N)(=O)=O)cc1C1=N[C@@](C)(c2ccc(Cl)cc2)[C@@](C)(c2ccc(Cl)cc2)N1C(=O)N1CCC(CC(N)=O)CC1]

In a manner analogous to the method described in Example 2, rac-(4S,5R)-2-(4-chloro-2-isopropoxy-5-sulfamoyl-phenyl)-4,5-bis-(4-chloro-phenyl)-4,5-dimethyl-4,5-dihydro-imidazole-1-carbonyl chloride was reacted with piperidine-4-yl-acetamide to give the tittle compound. LC-MS (ES, m/z) calculated for C₃₄H₃₉Cl₃N₅O₅S [(M+H)⁺] 734, observed 734

### Example 65

### 5-{(4S,5R)-4,5-Bis-(4-chloro-phenyl)-1-[4-(3-methanesulfonyl-propyl)-piperazine-1-carbonyl]-4,5-dimethyl-4,5-dihydro-1H-imidazol-2-yl}-2-chloro-4-isopropoxy-benzenesulfonamide

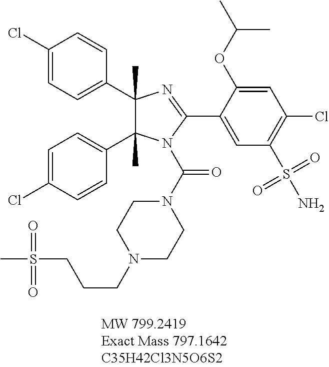 [SMILES: CC(C)Oc1cc(Cl)c(S(N)(=O)=O)cc1C1=N[C@@](C)(c2ccc(Cl)cc2)[C@@](C)(c2ccc(Cl)cc2)N1C(=O)N1CCN(CCCS(C)(=O)=O)CC1]

In a manner analogous to the method described in Example 2, rac-(4S,5R)-2-(4-chloro-2-isopropoxy-5-sulfamoyl-phenyl)-4,5-bis-(4-chloro-phenyl)-4,5-dimethyl-4,5-dihydro-imidazole-1-carbonyl chloride was reacted with 4-(3-methanesulfonyl-propyl)-piperazine (prepared as described in Ding et al. U.S. Pat. No. 7,851,626) to give the tittle compound. LC-MS (ES, m/z) calculated for C₃₅H₄₃Cl₃N₅O₆S₂ [(M+H)⁺] 798, observed 798.

### Example 66

### In Vitro Activity Assay

The ability of a test compound to inhibit the interaction between p53 and MDM2 protein was measured by an HTRF (homogeneous time-resolved fluorescence) assay in which recombinant GST-tagged MDM2 binds to a peptide that resembles the MDM2-interacting region of p53 Aca-SQETFSDLWKLLPEN-OH hereinafter p53-peptide). Binding of GST-MDM2 protein and p53-peptide (biotinylated on its N-terminal end) is registered by the FRET (fluorescence resonance energy transfer) between Europium (Eu)-labeled anti-GST antibody and streptavidin-conjugated Allophycocyanin (APC).

The test was performed in black flat-bottom 384-well plates (Costar) in a total volume of 40 uL containing: 90 nM biotinylated peptide, 160 ng/ml GST-MDM2, 20 nM streptavidin-APC (PerkinElmerWallac), 2 nM Eu-labeled anti-GST-antibody (PerkinElmerWallac), 0.02% bovine serum albumin (BSA), 1 mM dithiothreitol (DTT) and 20 mM Tris-borate saline (TBS) buffer as follows: add 10 uL of GST-MDM2 (640 ng/ml working solution) in reaction buffer to each well. Add 10 uL diluted compounds (1:5 dilution in reaction buffer) to each well, mix by shaking. Add 20 uL biotinylated p53 peptide (180 nM working solution) in reaction buffer to each well and mix on shaker. Incubate at 37° C. for 1 h. Add 20 uL streptavidin-APC and Eu-anti-GST antibody mixture (6 nM Eu-anti-GST and 60 nM streptavidin-APC working solution) in TBS buffer with 0.02% BSA, shake at room temperature for 30 minutes and read using a TRF-capable plate reader at 665 and 615 nm (Victor 5, Perkin ElmerWallac). If not specified, the reagents were purchased from Sigma Chemical Co.

The binding assay for GST-tagged MDMX to p53 peptide was the same as that of the MDM2 binding to p53 peptide except that the final concentration of GST-tagged MDMX was 25 nM.

Activity data for some of the exemplified test compounds expressed as IC50 (uM) is provided below in Table 1.

TABLE 1

| Example | MDMX IC50 (uM) | MDM2 IC50 (uM) |
|---|---|---|
| 2 | 1.0790 | 0.0057 |
| 3 | 1.1390 | 0.0054 |
| 4 | 1.7310 | 0.0054 |
| 5 | 3.6620 | 0.0153 |
| 6 | 1.2130 | 0.0066 |
| 7 | 2.4890 | 0.0063 |
| 8 | 1.0710 | 0.0044 |
| 9 | 1.7040 | 0.0058 |
| 10 | 0.9735 | 0.0042 |
| 11 | 1.2690 | 0.0045 |
| 12 | 0.9890 | 0.0055 |
| 13 | 1.3670 | 0.0061 |
| 14 | 1.4580 | 0.0047 |
| 15 | 1.1370 | 0.0055 |
| 16 | 0.8490 | 0.0050 |
| 17 | 1.3370 | 0.0052 |
| 18 | 1.4340 | 0.0060 |
| 19 | 3.7710 | 0.0125 |
| 20 | 3.6480 | 0.0054 |
| 21 | 1.4880 | 0.0058 |
| 22 | 1.3510 | 0.0046 |
| 23 | 1.8540 | 0.0055 |
| 24 | 1.5230 | 0.0072 |
| 25 | 1.5065 | 0.0044 |
| 26 | 2.0090 | 0.0052 |
| 27 | 2.8070 | 0.0088 |
| 28 | 1.4860 | 0.0046 |
| 29 | 1.0140 | 0.0050 |
| 30 | 3.3500 | 0.0127 |
| 31 | 1.5920 | 0.0053 |
| 32 | 1.4590 | 0.0052 |
| 33 | 1.1210 | 0.0053 |
| 34 | 1.3540 | 0.0063 |
| 35 | 2.8590 | 0.0067 |
| 36 | 1.2730 | 0.0048 |
| 37 | 3.9090 | 0.0061 |
| 38 | 2.8930 | 0.0084 |
| 39 | 1.0270 | 0.0061 |
| 40 | 0.2900 | 0.0061 |
| 41 | 0.1705 | 0.0069 |
| 42 | 6.6290 | 0.0125 |
| 43 | 1.3760 | 0.0097 |
| 44 | 1.5260 | 0.0139 |
| 45 | 1.1270 | 0.0129 |
| 46 | 1.8130 | 0.0122 |
| 47 | 1.0822 | 0.0066 |
| 48 | 1.3340 | 0.0117 |
| 49 | 1.3390 | 0.0087 |
| 50 | 1.4520 | 0.0093 |
| 51 | 0.7980 | 0.0070 |
| 52 | 3.7570 | 0.0173 |
| 53 | 1.1120 | 0.0145 |
| 54 | 4.0010 | 0.0161 |
| 55 | 3.9420 | 0.0181 |
| 56 | 3.4320 | 0.0085 |
| 57 | 1.6370 | 0.0400 |
| 58 | 0.2316 | 0.0046 |
| 59 | 0.1954 | 0.0058 |
| 60 | 0.5700 | 0.0150 |
| 61 | 0.2670 | 0.0091 |
| 62 | 0.1930 | 0.0048 |
| 63 | 0.2118 | 0.0044 |
| 64 | 0.2830 | 0.0062 |
| 65 | 0.3370 | 0.0077 |

What is claimed is:

1. A compound of Formula I 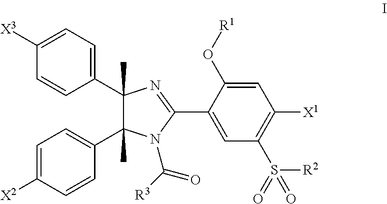
wherein,
X¹, X², X³ are the same or each is independently selected from halogen;
R¹ is lower alkyl that optionally is substituted with halogen;
R² is selected from the group
a) lower alkyl,
b) heterocycle, and
c) NR⁶R⁷;
R₃ is selected from the group:
a) -2-piperazinone that optionally is substituted with lower alkyl that optionally is substituted with OH,
b) a substituted piperazine of the formula II 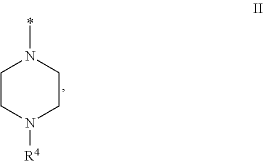
c) an optionally substituted piperidine of formula III 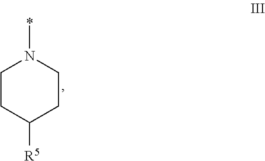
d) an optionally substituted pyrrolidynyl of formula IV 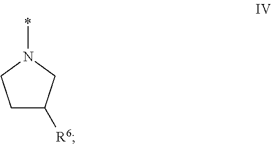
and
e) an optionally substituted piperidine of formula V 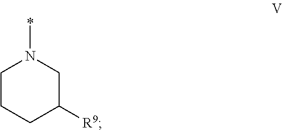
R⁴ is selected from the group:
a) H,
b) lower alkyl that optionally is substituted with
1) OR⁸,
2) C(O)NR⁶R⁷,
3) SO₂R⁸,
4) C(O)-heterocyclyl,
5) C(O)OR⁶,
6) NHSO₂R⁸,
7) NHC(O)R⁸, and
8) CF₃,
c) SO₂R⁸,
d) heterocyclyl substituted with oxo, and
e) C(O)R⁸;
R⁵ is selected from the group
a) H
b) lower alkyl that optionally is substituted with C(O)NR⁶R⁷, heterocyclyl and SO₂R⁸,
c) NR⁶R⁷,
e) NHC(O)NHR⁸,
f) NHC(O)-heterocyclyl,
g) NHSO₂R⁸
h) OR⁸,
i) heterocyclyl that optionally is substituted with oxo, and
j) piperidynyl which is spirally attached to the piperidine of formula III and which optionally is substituted with lower alkyl-C(O)NR⁶R⁷ or lower alkyl-SO₂R⁸;
R⁶ and R⁷ are the same or each is independently selected from
a) H,
b) lower alkyl that optionally is substituted with
1) O-lower alkyl,
2) N(CH₃)₂, and
3) heterocyclyl,
c) C(O)-lower alkyl; and
d) NR⁶R⁷—NR⁶R⁷;
R⁸ is selected from the group
a) H,
b) lower alkyl that optionally is substituted with OH and heteroaryl,
c) cycloalkyl, and
d) heterocyclyl that optionally is substituted with oxo; and
R⁹ is selected from H and OH;
or a pharmaceutically acceptable salt thereof.

2. The compound of claim 1 wherein X¹, X² and X³ are chloro, or a pharmaceutically acceptable salt of said compound.

3. The compound according to claim 2 wherein R¹ is ethyl or isopropyl, or a pharmaceutically acceptable salt of said compound.

4. The compound according to claim 3 wherein R² is lower alkyl, or a pharmaceutically acceptable salt of said compound.

5. The compound of claim 4 wherein R² is selected from methyl and ethyl.

6. The compound according to claim 3 wherein R² is heterocycle, or a pharmaceutically acceptable salt of said compound.

7. The compound of claim 6 wherein R² is selected from pyrrolidine, morpholine and 8-oxa-3-aza-bicyclo[3.2.1]octane, or a pharmaceutically acceptable salt of said compound.

8. The compound according to claim 3 wherein R² is NR⁶R⁷, or a pharmaceutically acceptable salt of said compound.

9. The compound of claim 8 wherein R² is selected from NH₂ and tert-butylamine, or a pharmaceutically acceptable salt of said compound.

10. The compound according to claim 9 wherein R³ is a piperazine of Formula II, or a pharmaceutically acceptable salt of said compound.

11. The compound of claim 10 wherein R⁴ Formula II is lower alkyl that is substituted with C(O)-heterocycle and the heterocycle is selected from 4-morpholinyl, 1-pyrrolidinyl and 1-piperidinyl.

12. The compound of claim 10 wherein R⁴ in Formula II is lower alkyl that is substituted with C(O)NR⁶R⁷, or a pharmaceutically acceptable salt of said compound.

13. The compound of claim 12 wherein NR⁶R⁷ is selected from NH₂ and N(CH₃)₂, or a pharmaceutically acceptable salt of said compound.

14. The compound of claim 10 wherein R⁴ in Formula II is lower alkyl that is substituted with SO₂R⁸, or a pharmaceutically acceptable salt of said compound.

15. The compound of claim 14 wherein R⁸ is lower alkyl selected from CH₃ and CH₂CH₃, or a pharmaceutically acceptable salt of said compound.

16. The compound of claim 10 wherein R⁴ in Formula II is SO₂R⁸, or a pharmaceutically acceptable salt of said compound.

17. The compound of claim 16 wherein R⁸ is methyl.

18. The compound of claim 10 wherein R⁴ in Formula II is heterocycle that optionally is substituted with oxo, or a pharmaceutically acceptable salt of said compound.

19. The compound of claim 18 wherein R⁴ is 3-tetrahydro-thiophenyl-1,1-dioxide.

20. The compound of claim 10 wherein R⁴ in Formula II is C(O)R⁸, or a pharmaceutically acceptable salt of said compound.

21. The compound of claim 20 wherein R⁸ is lower alkyl substituted by heteroaryl, or a pharmaceutically acceptable salt of said compound.

22. The compound according to claim 1 wherein R³ is a piperidine of Formula III, or a pharmaceutically acceptable salt of said compound.

23. The compound of claim 22 wherein R⁵ in Formula III is lower alkyl that optionally is substituted with a group selected from C(O)NR⁶R⁷ and SO₂R⁸, or a pharmaceutically acceptable salt of said compound.

24. The compound of claim 23 wherein R⁶ and R⁷ are both H, or a pharmaceutically acceptable salt of said compound.

25. The compound of claim 22 R⁵ in Formula III is NR⁶R⁷, or a pharmaceutically acceptable salt of said compound.

26. The compound of claim 25 wherein R⁵ is NH₂, or a pharmaceutically acceptable salt of said compound.

27. The compound of claim 22 wherein R⁵ in Formula III is NHC(O)NHR⁸, or a pharmaceutically acceptable salt of said compound.

28. The compound of claim 27 wherein R⁸ is lower alkyl selected from ethyl and propyl.

29. The compound of claim 22 wherein R⁵ in Formula III is NH(C)O-heterocycle, or a pharmaceutically acceptable salt of said compound.

30. The compound of claim 29 wherein the heterocycle is pyrrolidinyl.

31. The compound of claim 22 wherein R⁵ in Formula III is NHSO₂R⁸, or a pharmaceutically acceptable salt of said compound.

32. The compound of claim 31 wherein R⁸ is methyl, or a pharmaceutically acceptable salt of said compound.

33. The compound of claim 22 wherein R⁵ in Formula III is OR⁸, or a pharmaceutically acceptable salt of said compound.

34. The compound of claim 33 wherein R⁸ is H, or a pharmaceutically acceptable salt of said compound.

35. The compound of claim 22 wherein R⁵ in Formula III is heterocycle that optionally is substituted with oxo, or a pharmaceutically acceptable salt of said compound.

36. The compound of claim 35 wherein R⁵ is 5-oxo-[1,4]diazepam-1-yl.

37. The compound of claim 22 wherein R⁵ is piperidynyl that is spirally attached to the piperidine of Formula III and which optionally is substituted with —CH₂C(O)NH₂ or —(CH₂)₂SO₂CH₂CH₃, or a pharmaceutically acceptable salt of said compound.

38. The compound according to claim 9 wherein R³ is a pyrrolidynyl of Formula IV, or a pharmaceutically acceptable salt of said compound.

39. The compound of claim 28 wherein R⁶ in Formula IV is —N(lower alkyl)₂-N(lower alkyl)₂.

40. The compound according to claim 9 wherein R³ is an optionally substituted piperidine of Formula V, or a pharmaceutically acceptable salt of said compound.

41. The compound of claim 40 wherein and R⁹ in Formula V is OH, or a pharmaceutically acceptable salt of said compound.

42. The compound of claim 1 wherein X¹, X² and X³ are chloro, R¹ is ethyl or isopropyl, R² is lower alkyl or NR⁶R⁷, and R³ is an optionally substituted piperazine of Formula II, or a pharmaceutically acceptable salt of said compound.

43. The compound of claim 42 wherein R⁴ in Formula II is lower alkyl that optionally is substituted as defined in claim 1 or R⁴ SO₂R⁸, or a pharmaceutically acceptable salt of said compound.

44. The compound of claim 1 wherein X¹, X² and X³ are chloro, R¹ is ethyl or isopropyl, R² is lower alkyl or NR⁶R⁷, and R³ is an optionally substituted piperidine of Formula III, or a pharmaceutically acceptable salt of said compound.

45. The compound of claim 44 wherein R⁵ in Formula III is lower alkyl that optionally is substituted with C(O)NR⁶R⁷ or SO₂R⁸, or a pharmaceutically acceptable salt of said compound.

46. The compound of claim 44 wherein R⁵ in Formula III is NHSO₂R⁸, or a pharmaceutically acceptable salt of said compound.

47. The compound of claim 1 wherein X¹, X² and X³ are chloro, R¹ is ethyl or isopropyl, R² is lower alkyl or NR⁶R⁷, and R³ is an optionally substituted pyrrolidynyl of Formula IV, or a pharmaceutically acceptable salt of said compound.

48. The compound of claim 47 wherein R⁶ is N(lower alkyl)₂-N(lower alkyl)₂.

49. The compound of claim 1 wherein X¹, X² and X³ are chloro, R¹ is ethyl or isopropyl, R² is lower alkyl or NR⁶R⁷, and R³ an optionally substituted piperidine of Formula V, or a pharmaceutically acceptable salt of said compound.

50. The compound of claim 49 wherein R⁹ in Formula V is OH.

51. The compound of claim 1 wherein X¹, X² and X³ are chloro, R¹ is isopropyl, R² is methyl or NH-tert butyl, and R³ is an optionally substituted piperazine of Formula II, or a pharmaceutically acceptable salt of said compound.

52. The compound of claim 51 wherein R⁴ in Formula II is lower alkyl that optionally is substituted with SO₂R⁸ and R⁸ is methyl.

53. The compound of claim 1 wherein X¹, X² and X³ are chloro, R¹ is isopropyl, R² is methyl or pyrrolidine, R³ is an optionally substituted piperidine of Formula III, and R⁵ is methyl that is substituted with C(O)NH₂, or a pharmaceutically acceptable salt of said compound.

54. The compound of claim 1 wherein R³ is an optionally substituted piperazine of Formula II, said compound being selected from the group consisting of:
4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperazin-2-one;
[(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-[4-((S)-2,3-dihydroxy-propyl)-piperazin-1-yl]-methanone;
4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-1-((S)-3,4-dihydroxy-butyl)-piperazin-2-one;
2-{4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperazin-1-yl}-acetamide;
[(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-(4-methanesulfonyl-piperazin-1-yl)-methanone;
[(4S,5R)-2-(4-Chloro-5-ethanesulfonyl-2-ethoxy-phenyl)-4,5-bis-(4-chloro-phenyl)-4,5-dimethyl-4,5-dihydro-imidazol-1-yl]-[4-(3-methanesulfonyl-propyl)-piperazin-1-yl]-methanone;
[(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-[4-(3-ethanesulfonyl-propyl)-piperazin-1-yl]-methanone;
2-{4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperazin-1-yl}-N,N-bis-(2-methoxy-ethyl)-acetamide;
2-{4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperazin-1-yl}-N,N-bis-(2-ethoxy-ethyl)-acetamide;
2-{4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperazin-1-yl}-1-morpholin-4-yl-ethanone;
[(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-[4-(2-methanesulfonyl-ethyl)-piperazin-1-yl]-methanone;
[(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-[4-(1,1-dioxo-tetrahydro-thiophen-3-yl)-piperazin-1-yl]-methanone;
2-{4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperazin-1-yl}-N,N-bis-(2-isopropoxy-ethyl)-acetamide;
[(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-[4-(2-ethanesulfonyl-ethyl)-piperazin-1-yl]-methanone;
3-{4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperazin-1-yl}-propionamide;
2-{4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperazin-1-yl}-N-methyl-acetamide;
{4-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperazin-1-yl}-acetic acid, hydrochloride;
[(4S,5R)-2-(4-Chloro-2-isopropoxy-5-methanesulfonyl-phenyl)-4,5-bis-(4-chloro-phenyl)-4,5-dimethyl-4,5-dihydro-imidazol-1-yl]-[4-(3-methanesulfonyl-propyl)-piperazin-1-yl]-methanone;
[(4S,5R)-2-(4-Chloro-2-cyclopropylmethoxy-5-methanesulfonyl-phenyl)-4,5-bis-(4-chloro-phenyl)-4,5-dimethyl-4,5-dihydro-imidazol-1-yl]-[4-(3-methanesulfonyl-propyl)-piperazin-1-yl]-methanone;
[(4S,5R)-2-[4-Chloro-2-(2-fluoro-ethoxy)-5-methanesulfonyl-phenyl]-4,5-bis-(4-chloro-phenyl)-4,5-dimethyl-4,5-dihydro-imidazol-1-yl]-[4-(3-methanesulfonyl-propyl)-piperazin-1-yl]-methanone;
[(4S,5R)-2-(2-sec-Butoxy-4-chloro-5-methanesulfonyl-phenyl)-4,5-bis-(4-chloro-phenyl)-4,5-dimethyl-4,5-dihydro-imidazol-1-yl]-[4-(3-methanesulfonyl-propyl)-piperazin-1-yl]-methanone;
5-{(4S,5R)-4,5-Bis-(4-chloro-phenyl)-1-[4-(3,4-dihydroxy-butyl)-piperazine-1-carbonyl]-4,5-dimethyl-4,5-dihydro-1H-imidazol-2-yl}-N-tert-butyl-2-chloro-4-ethoxy-benzenesulfonamide;
5-((4S,5R)-4,5-Bis-(4-chloro-phenyl)-1-{4-[2-(2-hydroxy-ethoxy)-ethyl]-piperazine-1-carbonyl}-4,5-dimethyl-4,5-dihydro-1H-imidazol-2-yl)-N-tert-butyl-2-chloro-4-ethoxy-benzenesulfonamide;
5-((4S,5R)-4,5-Bis-(4-chloro-phenyl)-4,5-dimethyl-1-{4-[2-(4-methyl-piperazin-1-yl)-2-oxo-ethyl]-piperazine-1-carbonyl}-4,5-dihydro-1H-imidazol-2-yl)-N-tert-butyl-2-chloro-4-ethoxy-benzenesulfonamide;
2-{4-[(4S,5R)-2-(5-tert-Butylsulfamoyl-4-chloro-2-ethoxy-phenyl)-4,5-bis-(4-chloro-phenyl)-4,5-dimethyl-4,5-dihydro-imidazole-1-carbonyl]-piperazin-1-yl}-N-(tetrahydro-furan-2-ylmethyl)-acetamide;
2-{4-[(4S,5R)-2-(5-tert-Butylsulfamoyl-4-chloro-2-ethoxy-phenyl)-4,5-bis-(4-chloro-phenyl)-4,5-dimethyl-4,5-dihydro-imidazole-1-carbonyl]-piperazin-1-yl}-N-(2-methoxy-ethyl)-acetamide;
3-{4-[(4S,5R)-2-(5-tert-Butylsulfamoyl-4-chloro-2-ethoxy-phenyl)-4,5-bis-(4-chloro-phenyl)-4,5-dimethyl-4,5-dihydro-imidazole-1-carbonyl]-piperazin-1-yl}-propionamide;
5-{(4S,5R)-4,5-Bis-(4-chloro-phenyl)-1-[4-(1,1-dioxo-tetrahydro-thiophen-3-yl)-piperazine-1-carbonyl]-4,5-dimethyl-4,5-dihydro-1H-imidazol-2-yl}-N-tert-butyl-2-chloro-4-ethoxy-benzenesulfonamide;
5-{(4S,5R)-4,5-Bis-(4-chloro-phenyl)-4,5-dimethyl-1-[4-(2-tetrazol-1-yl-acetyl)-piperazine-1-carbonyl]-4,5-dihydro-1H-imidazol-2-yl}-N-tert-butyl-2-chloro-4-ethoxy-benzenesulfonamide;
{(4S,5R)-4,5-Bis-(4-chloro-phenyl)-2-[4-chloro-2-propoxy-5-(pyrrolidine-1-sulfonyl)-phenyl]-4,5-dimethyl-4,5-dihydro-imidazol-1-yl}-[4-(3-methanesulfonyl-propyl)-piperazin-1-yl]-methanone;
[(4S,5R)-2-[4-Chloro-2-ethoxy-5-(morpholine-4-sulfonyl)-phenyl]-4,5-bis-(4-chloro-phenyl)-4,5-dimethyl-4,5-dihydro-imidazol-1-yl]-[4-(3-methanesulfonyl-propyl)-piperazin-1-yl]-methanone;
[(4S,5R)-2-[4-Chloro-2-isopropoxy-5-(pyrrolidine-1-sulfonyl)-phenyl]-4,5-bis-(4-chloro-phenyl)-4,5-dimethyl-4,5-dihydro-imidazol-1-yl]-[4-(3-methanesulfonyl-propyl)-piperazin-1-yl]-methanone;
[(4S,5R)-2-{4-Chloro-2-isopropoxy-5-[(1R,5S)-(8-oxa-3-aza-bicyclo[3.2.1]oct-3-yl)sulfonyl]-phenyl}-4,5-bis-(4-chloro-phenyl)-4,5-dimethyl-4,5-dihydro-imidazol-1-yl]-[4-(3-methanesulfonyl-propyl)-piperazin-1-yl]-methanone;
5-{(4S,5R)-4,5-Bis-(4-chloro-phenyl)-1-[4-(3-methanesulfonyl-propyl)-piperazine-1-carbonyl]-4,5-dimethyl-4,5-dihydro-1H-imidazol-2-yl}-N-tert-butyl-2-chloro-4-isopropoxy-benzenesulfonamide; and
-{(4S,5R)-4,5-Bis-(4-chloro-phenyl)-1-[4-(3-methanesulfonyl-propyl)-piperazine-1-carbonyl]-4,5-dimethyl-4,5-dihydro-1H-imidazol-2-yl}-2-chloro-4-isopropoxy-benzenesulfonamide;
or a pharmaceutically acceptable salt of any of the foregoing compounds.

55. The compound of claim 1 wherein R³ is an optionally substituted piperidine of Formula III, said compound being selected from the group consisting of:
2-{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-acetamide;
(4-Amino-piperidin-1-yl)-[(4S,5R)-2-(4-chloro-2-ethoxy-5-methanesulfonyl-phenyl)-4,5-bis-(4-chloro-phenyl)-4,5-dimethyl-4,5-dihydro-imidazol-1-yl]-methanone;
[(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-(4-methanesulfonylmethyl-piperidin-1-yl)-methanone;
1-{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-3-ethyl-urea;
1-{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-3-isopropyl-urea;
[(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-[4-(2-methanesulfonyl-ethyl)-piperidin-1-yl]-methanone;
[(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-(3,9-diaza-spiro[5.5]undec-3-yl)-methanone;
2-{9-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-3,9-diaza-spiro[5.5]undec-3-yl}-acetamide;
[(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-[4-(3-methanesulfonyl-propyl)-piperidin-1-yl]-methanone;
2-{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-N,N-bis-(2-methoxy-ethyl)-acetamide;
2-{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-N,N-bis-(2-ethoxy-ethyl)-acetamide;
[(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-[9-(2-ethanesulfonyl-ethyl)-3,9-diaza-spiro[5.5]undec-3-yl]-methanone;
Pyrrolidine-1-carboxylic acid {1-[(4S,5R)-2-(4-chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-amide;
1-{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-3-cyclopentyl-urea;
{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-urea;
N-{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-methanesulfonamide;
1-{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-3-(1,1-dioxo-tetrahydro-1%6-thiophen-3-yl)-urea;
N-{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-acetamide;
[(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-(4-hydroxy-piperidin-1-yl)-methanone;
2-{1-[(4S,5R)-2-(4-Chloro-2-ethoxy-5-methanesulfonyl-phenyl)-4,5-bis-(4-chloro-phenyl)-4,5-dimethyl-4,5-dihydro-imidazole-1-carbonyl]-piperidin-4-yl}-acetamide;
2-{1-[(4S,5R)-2-(4-Chloro-2-isopropoxy-5-methanesulfonyl-phenyl)-4,5-bis-(4-chloro-phenyl)-4,5-dimethyl-4,5-dihydro-imidazole-1-carbonyl]-piperidin-4-yl}-acetamide;
5-{(4S,5R)-4,5-Bis-(4-chloro-phenyl)-4,5-dimethyl-1-[4-(5-oxo-[1,4]diazepan-1-yl)-piperidine-1-carbonyl]-4,5-dihydro-1H-imidazol-2-yl}-N-tert-butyl-2-chloro-4-ethoxy-benzenesulfonamide;
2-{1-[(4S,5R)-2-[4-Chloro-2-ethoxy-5-(pyrrolidine-1-sulfonyl)-phenyl]-4,5-bis-(4-chloro-phenyl)-4,5-dimethyl-4,5-dihydro-imidazole-1-carbonyl]-piperidin-4-yl}-acetamide;
2-{1-[(4S,5R)-2-[4-Chloro-2-isopropoxy-5-(pyrrolidine-1-sulfonyl)-phenyl]-4,5-bis-(4-chloro-phenyl)-4,5-dimethyl-4,5-dihydro-imidazole-1-carbonyl]-piperidin-4-yl}-acetamide;
2-{1-[(4S,5R)-2-(5-tert-Butylsulfamoyl-4-chloro-2-isopropoxy-phenyl)-4,5-bis-(4-chloro-phenyl)-4,5-dimethyl-4,5-dihydro-imidazole-1-carbonyl]-piperidin-4-yl}-acetamide;
2-{1-[(4S,5R)-2-{4-Chloro-2-isopropoxy-5-[(1R,5S)-(8-oxa-3-aza-bicyclo[3.2.1]oct-3-yl)sulfonyl]-phenyl}-4,5-bis-(4-chloro-phenyl)-4,5-dimethyl-4,5-dihydro-imidazole-1-carbonyl]-piperidin-4-yl}-acetamide; and
2-{1-[(4S,5R)-2-(4-Chloro-2-isopropoxy-5-sulfamoyl-phenyl)-4,5-bis-(4-chloro-phenyl)-4,5-dimethyl-4,5-dihydro-imidazole-1-carbonyl]-piperidin-4-yl}-acetamide;
or a pharmaceutically acceptable salt of any of the foregoing compounds.

56. The compound of claim 1 wherein R³ is a group of Formula IV or V, said compound being selected from the group consisting of:
[(4S,5R)-2-(4-Chloro-2-ethoxy-5-methanesulfonyl-phenyl)-4,5-bis-(4-chloro-phenyl)-4,5-dimethyl-4,5-dihydro-imidazol-1-yl]-{3-[(3-dimethylamino-propyl)-methyl-amino]-pyrrolidin-1-yl}-methanone; and
5-[(4S,5R)-4,5-Bis-(4-chloro-phenyl)-1-(3-hydroxy-piperidine-1-carbonyl)-4,5-dimethyl-4,5-dihydro-1H-imidazol-2-yl]-N-tert-butyl-2-chloro-4-ethoxy-benzenesulfonamide; or a pharmaceutically acceptable salt thereof.

57. A compound selected from the group consisting of:
[(4S,5R)-2-(4-Chloro-2-isopropoxy-5-methanesulfonyl-phenyl)-4,5-bis-(4-chloro-phenyl)-4,5-dimethyl-4,5-dihydro-imidazol-1-yl]-[4-(3-methanesulfonyl-propyl)-piperazin-1-yl]-methanone;
2-{1-[(4S,5R)-2-(4-Chloro-2-isopropoxy-5-methanesulfonyl-phenyl)-4,5-bis-(4-chloro-phenyl)-4,5-dimethyl-4,5-dihydro-imidazole-1-carbonyl]-piperidin-4-yl}-acetamide;
2-{1-[(4S,5R)-2-[4-Chloro-2-isopropoxy-5-(pyrrolidine-1-sulfonyl)-phenyl]-4,5-bis-(4-chloro-phenyl)-4,5-dimethyl-4,5-dihydro-imidazole-1-carbonyl]-piperidin-4-yl}-acetamide; and
5-{(4S,5R)-4,5-Bis-(4-chloro-phenyl)-1-[4-(3-methanesulfonyl-propyl)-piperazine-1-carbonyl]-4,5-dimethyl-4,5-dihydro-1H-imidazol-2-yl}-N-tert-butyl-2-chloro-4-isopropoxy-benzenesulfonamide;
or a pharmaceutically acceptable salt of any of the foregoing compounds.

58. A pharmaceutical composition comprising the compounds of claim 1, or a pharmaceutically acceptable salt thereof, as an active ingredient together with a pharmaceutically acceptable carrier or excipient.

59. A method of treating or ameliorating cancer comprising administering to a subject in need of such treatment a therapeutically effective amount of a compound of claim 1.
